(19) United States
(12) Patent Application Publication
(10) Pub. No.: US 2011/0200531 A1
(43) Pub. Date: Aug. 18, 2011

(54) TREATMENT AND DIAGNOSIS OF CENTRAL NERVOUS SYSTEM DISORDERS

(75) Inventor: Zhiqun Tan, Irvine, CA (US)

(73) Assignee: THE REGENTS OF THE UNIVERSITY OF CALIFORNIA, Oakland, CA (US)

(21) Appl. No.: 12/882,071

(22) Filed: Sep. 14, 2010

Related U.S. Application Data
(60) Provisional application No. 61/242,289, filed on Sep. 14, 2009.

(51) Int. Cl. A61K 49/00; A61K 31/4965; A61K 31/55; A61P 25/28; A61P 27/06; A61P 43/00

(52) U.S. Cl. 424/9.2; 514/252.1; 514/215; 424/9.1

(57) ABSTRACT

The invention relates to the use of tetramethylpyrazine (TMP) for the treatment of patients at risk for the development of, or diagnosed as having, CNS disorders such as Alzheimer's disease (AD), Parkinson's disease (PD), glaucoma, and Huntington's Disease (HD), as well as traumatic brain injury (TBI). The present invention also relates to the use of retinal imaging to diagnose and monitor efficacy of treatments for such CNS disorders.

### CROSS-REFERENCE TO RELATED APPLICATIONS

This application claims the benefit of U.S. provisional application No. 61/242,289, filed Sep. 14, 2009, which is hereby incorporated by reference in its entirety.

### 1. INTRODUCTION

The invention relates to the use of tetramethylpyrazine (TMP) for the treatment of patients at risk for the development of CNS disorders such as Alzheimer's disease (AD), Parkinson's disease (PD), glaucoma, and Huntington's Disease (HD). The invention relates to the use of tetramethylpyrazine (TMP) for the treatment of patients diagnosed as having CNS disorders such as Alzheimer's disease (AD), Parkinson's disease (PD), glaucoma, and Huntington's Disease (HD), as well as traumatic brain injury (TBI). The present invention relates to the use of retinal imaging to diagnose and monitor efficacy of treatments for CNS disorders such as Alzheimer's disease (AD), Parkinson's disease (PD), and Huntington's Disease (HD), glaucoma, as well as traumatic brain injury (TBI).

### 2. BACKGROUND

Degenerative disorders of the central nervous system (CNS) comprise a heterogeneous group of disorders that are typically of unknown etiology and pathogenesis. At present, there exists no cure for CNS disorders such as Alzheimer's disease (AD), Parkinson's disease (PD), and Huntington's Disease (HD), as well as traumatic brain injury (TBI) and glaucoma. Indeed, in most instances, available treatments for CNS diseases offer relatively small symptomatic benefit but remain palliative in nature. As such, there remains a need for effective drugs for use in the treatment of CNS disorders.

Tetramethylpyrazine (TMP), also known as ligustrazine, is a drug originally isolated from the rhizome of Ligusticum wallichii, a flowering plant in the carrot family known for its use in traditional Chinese medicine that is commonly called chuanxiong.

### 3. SUMMARY

Presented herein are methods of treating patients having or at risk of developing degenerative disorders of the CNS, e.g., Alzheimer's disease (AD), using a tetramethylpyrazine compound (e.g., TMP). In certain embodiments TMP is administered to a patient in need of such treatment (i.e., a patient that has been diagnosed as being in need of such treatment). In certain embodiments, TMP is administered as a single-agent therapy to a patient in need of such treatment. In other embodiments, TMP can be administered in combination with one or more additional therapies to a patient in need of such treatment. Such therapies may include the use of drugs used in the treatment of degenerative disorders of the CNS, e.g., AD, mild cognitive impairment (MCI), Parkinson's disease (PD), glaucoma, Huntington's disease (HD), and/or traumatic brain injury (TBI).

In certain embodiments, the administration of TMP to a patient having or at risk of developing a degenerative disorder of the CNS results in the inhibition or reduction of one or more pathological conditions associated with the degenerative disorder of the CNS. In certain embodiments, the administration of TMP to a patient having or at risk of developing a degenerative disorder of the CNS, e.g., AD, results in the inhibition or reduction one or more of the following pathologies: accumulation of protein aggregates (e.g., amyloid plaques or neurofibrillary tangles (NFT)); inflammatory events; cerebral amyloid angiopathy; oxidative stress; mitochondrial stress; endoplasmic reticulum stress; disruption of axonal transport; loss of spines and/or synapses; cholinergic dysfunction; neuritic fragmentation; loss of estrogen; loss of neurotrophic factors; loss of neurons.

In one aspect of the methods presented herein, TMP is administered to the patient orally. In another aspect of the methods presented herein, TMP is administered to the patient subcutaneously (s.c.). In another aspect of the methods presented herein, TMP is administered to the patient intraocularly, e.g., via eye drops or injection. In another aspect of the methods presented herein, TMP is administered to the patient as a topical opthalmic. In another aspect of the methods presented herein, TMP is administered to the patient intranasally (i.n.). In a specific embodiment, TMP is administered to the patient at a dose of 300 mg/kg/day. In another specific embodiment, TMP is administered to the patient orally at a dose of 300 mg/kg/day. In another specific embodiment, TMP is administered to the patient at a dose of 50 mg/kg/day. In another specific embodiment, TMP is administered to the patient subcutaneously (s.c.) at a dose of 50 mg/kg/day.

Any degenerative disorder of the CNS can be treated in accordance with the methods provided herein including, but not limited to, AD, PD, HD, glaucoma, TBI, MCI, age-related macular degeneration, epilepsy, retinal diseases associated with Alzheimer's disease and other retinal diseases, demyelinating diseases of the CNS, multiple sclerosis (MS), transverse myelitis, optic neuritis, Devic's disease, demyelinating diseases of the peripheral nervous system, Guillain-Barré syndrome, chronic inflammatory demyelinating polyneuropathy, hemiplegia, cerebral palsy, paraplegia, quadraplegia, vascular dementia, cerebral amyloid deposits, bacterial and viral meningitis, cerebral toxoplasmosis, brain cancers (e.g., metastatic carcinoma of the brain, glioblastoma, astrocytoma, and acoustic neuroma), hydrocephalus, and encephalitis.

In one embodiment, provided herein is a method for treating a degenerative disorder of the CNS in a patient in need thereof comprising administering a therapeutically effective amount of TMP. In another embodiment, provided herein is a method for treating AD in a patient in need thereof comprising administering a therapeutically effective amount of TMP. In another embodiment, provided herein is a method of treating MCI in a patient in need thereof comprising administering a therapeutically effective amount of TMP. In another embodiment, provided herein is a method of treating PD in a patient in need thereof comprising administering a therapeutically effective amount of TMP. In another embodiment, provided herein is a method of treating HD in a patient in need thereof comprising administering a therapeutically effective amount of TMP. In another embodiment, provided herein is a method of treating glaucoma in a patient in need thereof comprising administering a therapeutically effective amount of TMP. In yet another embodiment, presented herein is a method treating TBI in a patient in need thereof comprising administering a therapeutically effective amount of TMP.

In a specific embodiment, presented herein is a method for preventing the onset of AD in a patient at risk of developing AD, comprising administering a therapeutically effective amount of TMP to the patient at risk of developing AD. In another specific embodiment, presented herein is a method for treating early onset AD comprising administering a therapeutically effective amount of TMP to a patient with early onset AD. In another specific embodiment, presented herein is a method for treating familial AD comprising administering a therapeutically effective amount of TMP to a patient with familial AD. In another specific embodiment, presented herein is a method for treating mild AD comprising administering a therapeutically effective amount of TMP to a patient with mild AD. In another specific embodiment, presented herein is a method for treating MCI associated with AD comprising administering a therapeutically effective amount of TMP to a patient with MCI associated with AD. In another specific embodiment, presented herein is a method for treating moderate AD comprising administering a therapeutically effective amount of TMP to a patient with moderate AD. In another specific embodiment, presented herein is a method for treating severe AD comprising administering a therapeutically effective amount of TMP to a patient with severe AD. In another specific embodiment, presented herein is a method for treating late-stage AD comprising administering a therapeutically effective amount of TMP to a patient with late-stage AD. In one aspect of these embodiments, TMP is administered to the patient at a dose of 300 mg/kg/day. In another aspect of these embodiments, TMP is administered to the patient orally at a dose of 300 mg/kg/day. In another aspect of these embodiments, TMP is administered to the patient at a dose of 50 mg/kg/day. In another aspect of these embodiments, TMP is administered to the patient s.c. at a dose of 50 mg/kg/day.

The invention further relates to methods utilizing retinal imaging to diagnose and monitor efficacy of treatments for CNS disorders such as AD, PD, glaucoma, and HD, as well as TBI. In a particular embodiment, the invention relates to methods utilizing retinal imaging to diagnose and monitor efficacy of treatments for AD. In one embodiment retinal imaging is performed on a patient to determine levels of pathological changes in the retina to diagnose that the patient has a CNS disorder, such as AD, PD, and HD, in particular AD. In accordance with the present invention retinal imaging is performed on a patient prior to the onset of the therapeutic regimen and during or at the completion of the therapeutic regimen, wherein an improvement in the pathology indicates that the therapy is effective. In accordance with the present invention, determining the presence of Aβ plaques and amyloid angiopathy in the retina of a patient may be used to diagnose and monitor efficacy of therapeutic methods. A decrease in the presence of Aβ plaques in the retina indicates that the therapeutic regimen is effective. An improvement in amyloid angiopathy in the retina indicates that the therapeutic regimen is effective. In accordance with the present invention, determining improvement in retinal pathology, including a decrease in retinal thickness, indicates that the therapeutic regimen is effective.

The invention further relates to methods utilizing retinal imaging to diagnose and monitor efficacy of treating CNS disorders such as AD, PD, glaucoma, and HD, as well as TBI, by administrating an effective amount of a tetramethylpyrazine compound (e.g., TMP). In a particular embodiment, the invention relates to methods utilizing retinal imaging to diagnose and monitor efficacy of treating AD, by administrating effective amounts of TMP. In accordance with the present invention, determining the presence of Aβ plaques and amyloid angiopathy in the retina of a patient may be used to diagnose and monitor efficacy of administration of TMP. A decrease in the presence of Aβ plaques in the retina indicates that the TMP therapeutic regimen is effective. An improvement in amyloid angiopathy in the retina indicates that the TMP therapeutic regimen is effective. In accordance with the present invention, determining improvement in retinal pathology, including a decrease in retinal thickness, indicates that the TMP therapeutic regimen is effective.

In another aspect, presented herein are methods for preventing the onset of a retinal disease in a patient at risk of developing a retinal disease, comprising administering a therapeutically effective amount of a tetramethylpyrazine compound (e.g., TMP) to the patient at risk of developing a retinal disease. In another aspect, presented herein are methods for treating a retinal disease in a patient having a retinal disease, comprising administering a therapeutically effective amount of a tetramethylpyrazine compound (e.g., TMP) to the patient having a retinal disease. Exemplary retinal diseases that can be prevented and/or treated in accordance with the methods provided herein include age-related macular degeneration (AMD); wet age-related macular degeneration; dry age-related macular degeneration; Bardet-Biedl Syndrome; Best vitelliform macular dystrophy (BVMD) or Best's disease; cataracts; central retinal vein occlusion (neovascular glaucoma); cone-rod dystrophy; degenerative myopia; diabetic retinopathy (DR); diabetic papillopathy; Doyne honeycomb retinal dystrophy; epiretinal membrane, also known as: macular pucker, macular wrinkling, or cellophane membrane; erosive vitreoretinopathy; glaucoma; juvenile-onset macular degeneration; macular degeneration; wet macular degeneration; dry macular degeneration; macular dystrophy; macular hole; morning glory anomaly; open angle glaucoma; closed angle glaucoma; optic nerve drusen; optic nerve coloboma; proliferative vitreoretinopathy; pseudotumor cerebri; retinal degeneration; retinal detachment; retinal dystrophy; retinal tear; retinis pigmentosa; retinal drusen; Type 2 Leber congenital amaurosis; uveitic glaucoma; diseases of the retinal pigment epithelium (RPE); pseudoexfoliation; pigment dispersion syndrome, phacolytic glaucoma; lens particle glaucoma; chorioretinal folds; rhegmatogenous detachment; angloid streaks; tractional detachment; degenerative retinoschisis.

In a specific embodiment, presented herein are methods for preventing the onset of glaucoma in a patient at risk of developing glaucoma, comprising administering a therapeutically effective amount of a tetramethylpyrazine compound (e.g., TMP) to the patient at risk of developing glaucoma. In a specific embodiment, presented herein is a method for preventing early onset glaucoma in a patient in need thereof, comprising administering a therapeutically effective amount of TMP to a patient with early onset glaucoma. In another specific embodiment, presented herein is a method for preventing the onset of open-angle glaucoma in a patient at risk of developing open-angle glaucoma, comprising administering a therapeutically effective amount of TMP to the patient at risk of developing open-angle glaucoma. In another specific embodiment, presented herein is a method for preventing the onset of open-angle glaucoma comprising administering a therapeutically effective amount of TMP to a patient with early onset open-angle glaucoma. In another specific embodiment, presented herein is a method for preventing glaucoma comprising administering a therapeutically effective amount of TMP to a patient with decreased cerebrospinal fluid pressure. In another specific embodiment, presented herein is a method for preventing glaucoma comprising administering a therapeutically effective amount of TMP to a patient with ocular hypertension. In another specific embodiment, presented herein is a method for preventing glaucoma comprising administering a therapeutically effective amount of TMP to a patient with increased loss of retinal ganglion cells. In another specific embodiment, presented herein is a method for preventing glaucoma comprising administering a therapeutically effective amount of TMP to a patient with atrophy of the optic nerve. In another specific embodiment, presented herein is a method for preventing glaucoma comprising administering a therapeutically effective amount of TMP to a patient with loss in visual field. In some embodiments, TMP is administered to the patient via eye drops. In other embodiments, TMP is administered to the patient as a topical ophthalmic solution.

In another specific embodiment, presented herein are methods for treating glaucoma in a patient in need of such treatment, comprising administering a therapeutically effective amount of a tetramethylpyrazine compound (e.g., TMP) to a patient with glaucoma. In a specific embodiment, presented herein is a method for treating glaucoma comprising administering a therapeutically effective amount of TMP to a patient with glaucoma, wherein the treatment prevents or reduces decreased cerebrospinal fluid pressure. In another specific embodiment, presented herein is a method for treating glaucoma comprising administering a therapeutically effective amount of TMP to a patient with glaucoma, wherein the treatment prevents or reduces ocular hypertension. In another specific embodiment, presented herein is a method for treating glaucoma comprising administering a therapeutically effective amount of TMP to a patient with glaucoma, wherein the treatment prevents or reduces the loss of retinal ganglion cells. In another specific embodiment, presented herein is a method for treating glaucoma comprising administering a therapeutically effective amount of TMP to a patient with glaucoma, wherein the treatment prevents or reduces the neurodenegeration of retinal ganglion cells. In another specific embodiment, presented herein is a method for treating glaucoma comprising administering a therapeutically effective amount of TMP to a patient with glaucoma, wherein the treatment prevents or reduces atrophy of the optic nerve. In another specific embodiment, presented herein is a method for treating glaucoma comprising administering a therapeutically effective amount of TMP to a patient with glaucoma, wherein the treatment prevents or reduces a loss in visual field. In another specific embodiment, presented herein is a method for treating glaucoma comprising administering a therapeutically effective amount of TMP to a patient with glaucoma, wherein the treatment prserves the visual field. In some embodiments, TMP is administered to the patient with glaucoma via eye drops. In other embodiments, TMP is administered to the patient with glaucoma as a topical ophthalmic solution.

In some embodiments, the methods for treating glaucoma presented herein involve the administration of TMP as a single-agent therapy, to a patient in need thereof. In a specific embodiment, presented herein is a method for treating glaucoma comprising administering to a patient in need thereof a therapeutically effective amount of TMP as a single agent. In another specific embodiment, presented herein is a method for treating glaucoma comprising administering to a patient in need thereof a pharmaceutical composition comprising TMP, as the single active ingredient, and a pharmaceutically acceptable carrier, excipient or vehicle. In a some embodiments, TMP is administered to the patient with glaucoma via eye drops. In other embodiments TMP is administered to the patient with glaucoma as a topical ophthalmic solution.

### 3.1 Terminology

Unless specified otherwise, as used hereinafter, the term “degenerative disorder of the CNS” refers to any disease or disorder of the central nervous system that is characterized by the degeneration of, loss of, reduction in function of, or disruption of one or more components of the central nervous system, e.g., the loss/degeneration of neurons.

As used herein, the term “therapeutically effective amount” in the context of administering TMP to a patient having or at risk of developing a degenerative disorder of the CNS refers to the amount of TMP that results in a beneficial or therapeutic effect. In specific embodiments, a “therapeutically effective amount” of TMP refers to an amount of TMP which is sufficient to achieve at least one, two, three, four or more of the following effects: (i) the reduction or amelioration of the severity of a degenerative disease of the CNS and/or one or more symptoms associated therewith; (ii) the reduction in the duration of one or more symptoms associated with a degenerative disease of the CNS; (iii) the prevention in the recurrence of a degenerative disease of the CNS or one or more symptoms associated with a degenerative disease of the CNS; (iv) the regression of a degenerative disease of the CNS and/or one or more symptoms associated therewith; (v) the reduction in hospitalization of a patient having a degenerative disease of the CNS; (vi) the reduction in hospitalization length of a patient having a degenerative disease of the CNS; (vii) the increase in the survival of a patient having a degenerative disease of the CNS; (viii) the inhibition of the progression of a degenerative disease of the CNS and/or one or more symptoms associated therewith; (ix) the enhancement or improvement of the therapeutic effect of another therapy; (x) a decrease in hospitalization rate of a patient having a degenerative disease of the CNS; (xi) the reduction in the number of symptoms associated with a degenerative disease of the CNS in a patient having a degenerative disease of the CNS; (xii) an increase in symptom-free survival of a patient having a degenerative disease of the CNS; (xiii) a reduction in the accumulation of protein aggregates (e.g., amyloid plaques or neurofibrillary tangles (NFT)); (xiv) a reduction in inflammatory events associated with a degenerative disease of the CNS; (xv) a reduction in cerebral amyloid angiopathy associated with a degenerative disease of the CNS; (xvi) a reduction in oxidative stress associated with a degenerative disease of the CNS; (xvii) a reduction in mitochondrial stress associated with a degenerative disease of the CNS; (xviii) a reduction in endoplasmic reticulum stress associated with a degenerative disease of the CNS; (xix) a reduction in the disruption of axonal transport associated with a degenerative disease of the CNS; (xx) a reduction in the loss of spines and/or synapses; (xxi) a reduction in cholinergic dysfunction associated with a degenerative disease of the CNS; (xxii) a reduction in neuritic fragmentation associated with a degenerative disease of the CNS; (xxiii) a reduction in the loss of estrogen associated with a degenerative disease of the CNS; (xxiv) a reduction in the loss of neurotrophic factors associated with a degenerative disease of the CNS; and/or (xxv) a reduction in the loss of neurons associated with a degenerative disease of the CNS.

As used herein, the term “elderly” refers to a human 65 years or older.

As used herein, the term “adult” refers to a human that is 18 years or older.

As used herein, the term “middle-aged” refers to a human between the ages of 30 and 64.

As used herein, the terms “therapies” and “therapy” can refer to any protocol(s), method(s), compositions, formulations, and/or agent(s) that can be used in the prevention, treatment, management, or amelioration of a condition or disorder or symptom thereof. In certain embodiments, the terms “therapies” and “therapy” refer to drug therapy, adjuvant therapy, radiation, surgery, biological therapy, supportive therapy, and/or other therapies useful in treatment, management, prevention, or amelioration of a condition or disorder or one or more symptoms thereof. In certain embodiments, the term “therapy” refers to a therapy other than TMP or a pharmaceutical composition thereof. In specific embodiments, an “additional therapy” and “additional therapies” refer to a therapy other than a treatment using TMP or pharmaceutical composition. In a specific embodiment, a therapy includes the use of TMP as an adjuvant therapy. For example, using TMP in conjunction with a drug therapy, biological therapy, surgery, and/or supportive therapy.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the term “about” means a range around a given value wherein the resulting value is substantially the same as the expressly recited value. In one embodiment, “about” means within 25% of a given value or range. For example, the phrase “about 70% by weight” comprises at least all values from 52% to 88% by weight. In another embodiment, the term “about” means within 10% of a given value or range. For example, the phrase “about 70% by weight” comprises at least all values from 63% to 77% by weight. In another embodiment, the term “about” means within 7% of a given value or range. For example, the phrase “about 70% by weight” comprises at least all values from 65% to 75% by weight.

As used herein, the term “small molecule” and analogous terms include, but are not limited to, peptides, peptidomimetics, amino acids, amino acid analogs, polynucleotides, polynucleotide analogs, nucleotides, nucleotide analogs, other organic and inorganic compounds (I.e., including heteroorganic and organometallic compounds) and forms thereof having a molecular weight of less than about 10,000 grams per mole, or less than about 5,000 grams per mole, or less than about 1,000 grams per mole, or less than about 500 grams per mole, or less than about 100 grams per mole.

As used herein, the term “in combination,” in the context of the administration of TMP, refers to the administration of TMP and one or more additional compounds or agents. The use of the term “in combination” does not restrict the order of administration of TMP and the one or more additional compounds or agents.

As used herein, the term “combination product” refers to a product comprising TMP and one or more additional compounds or agents.

As used herein, the term “isolated,” as it refers to TMP, means the physical state of TMP after being separated and/or purified from precursors and other substances found in a synthetic process (e.g., from a reaction mixture) or natural source or combination thereof according to a process or processes described herein or which are well known to the skilled artisan (e.g., chromatography, recrystallization and the like) in sufficient purity to be capable of characterization by standard analytical techniques described herein or well known to the skilled artisan. In a specific embodiment, TMP is at least 60% pure, at least 65% pure, at least 70% pure, at least 75% pure, at least 80% pure, at least 85% pure, at least 90% pure, at least 99% pure, or at least 99.9% pure as assessed by techniques known to one of skill in the art.

As used herein, the terms “subject” and “patient” are used interchangeably, and refer to an animal (e.g., birds, reptiles, and mammals), such as a mammal including a non-primate (e.g., a camel, donkey, zebra, cow, pig, horse, goat, sheep, cat, dog, rat, and mouse) and a primate (e.g., a monkey, chimpanzee, and a human). In a specific embodiment, the subject is a human.

As used herein, the terms “treat,” “treatment,” and “treating” in the context of administration of TMP to a subject to treat a degenerative disease of the CNS, refer to a therapeutic effect achieved following the administration of TMP or a combination TMP and one or more additional compounds or agents. In a specific embodiment, the therapeutic effect is at least one or more of the following effects resulting from the administration of TMP or a combination TMP and one or more additional compounds or agents: (i) the reduction or amelioration of the severity of a degenerative disease of the CNS and/or one or more symptoms associated therewith; (ii) the reduction in the duration of one or more symptoms associated with a degenerative disease of the CNS; (iii) the prevention in the recurrence of a degenerative disease of the CNS or one or more symptoms associated with a degenerative disease of the CNS; (iv) the regression of a degenerative disease of the CNS and/or one or more symptoms associated therewith; (v) the reduction in hospitalization of a patient having a degenerative disease of the CNS; (vi) the reduction in hospitalization length of a patient having a degenerative disease of the CNS; (vii) the increase in the survival of a patient having a degenerative disease of the CNS; (viii) the inhibition of the progression of a degenerative disease of the CNS and/or one or more symptoms associated therewith; (ix) the enhancement or improvement of the therapeutic effect of another therapy; (x) a decrease in hospitalization rate of a patient having a degenerative disease of the CNS; (xi) the reduction in the number of symptoms associated with a degenerative disease of the CNS in a patient having a degenerative disease of the CNS; (xii) an increase in symptom-free survival of a patient having a degenerative disease of the CNS; (xiii) a reduction in the accumulation of protein aggregates (e.g., amyloid plaques or neurofibrillary tangles (NFT)); (xiv) a reduction in inflammatory events associated with a degenerative disease of the CNS; (xv) a reduction in cerebral amyloid angiopathy associated with a degenerative disease of the CNS; (xvi) a reduction in oxidative stress associated with a degenerative disease of the CNS; (xvii) a reduction in mitochondrial stress associated with a degenerative disease of the CNS; (xviii) a reduction in endoplasmic reticulum stress associated with a degenerative disease of the CNS; (xix) a reduction in the disruption of axonal transport associated with a degenerative disease of the CNS; (xx) a reduction in the loss of spines and/or synapses; (xxi) a reduction in cholinergic dysfunction associated with a degenerative disease of the CNS; (xxii) a reduction in neuritic fragmentation associated with a degenerative disease of the CNS; (xxiii) a reduction in the loss of estrogen associated with a degenerative disease of the CNS; (xxiv) a reduction in the loss of neurotrophic factors associated with a degenerative disease of the CNS; and/or (xxv) a reduction in the loss of neurons associated with a degenerative disease of the CNS.

As used herein, the term “a patient at risk for development of a CNS disorder” refers to a patient that has been diagnosed as having certain risk factors associated with the development of a CNS disorder such as Alzheimer's disease (AD), Parkinson's disease (PD), and Huntington's Disease (HD). Exemplary risk factors associated with the development of AD include: alterations in neural function as observed by functional magnetic resonance imaging (fMRI); amyloid deposition as measured by PiB (Pittsburgh compound B); an increase in PiB imaging over time; an increase in PiB imaging relative to controls; an increase in PiB staining over time; an increase in PiB staining relative to controls; an increase in PiB uptake over time; an increase in PiB uptake relative to controls; plaque formation as measured by PiB; compromised brain function in areas including the hippocampus, temporo-parietal cortex, and posterior cingulated gyms; constant levels or decreases in synaptic density, neurogenesis, brain network efficiency and flexibility, or other experience-based brain changes; cortical infarcts; diabetes (in particular type 2 diabetes and hyperinsulinemia) which can be defined by glycated hemoglobin 6.5%, fast plasma glucose above 126 mg/dl, 3.2 h plasma glucose 200 mg/dl during oral glucose tolerance test, or random plasma glucose above 200 mg/dl in a patient with clinical symptoms of hyperglycemia; family history of Alzheimer's disease; glucose dysregulation; cerebral blood flow (CBF) deficits; decreased oxygen and glucose supply to the brain; increased GSK-3 (glycogen synthase kinase-3) activity; increased homocysteine (HCY) levels; renal insufficiency; exposure to certain drugs such as methotrexate; insulin resistance; medial temporal-lobe (MTL) atrophy as measured by magnetic resonance imaging (MRI); mild cognitive impairment (MCI); reductions in regional glucose metabolism as measured by fluorodeoxyglucose (FDG) positron emission tomography (PET); subcortical infarcts; white-matter hyperintensities (WMHIs). Other exemplary risk factors associated with the development of AD include Alzheimer's disease-related polymorphisms, or combinations of these polymorphisms, of the following genes: ACE (Angiotensin-converting enzyme; Polymorphism: rs1800764); APOE (Apolipoprotein E; Polymorphisms: apoe ε2, apoe ε3, apoe ε4); CHRNB2 (Cholinergic receptor, nicotinic, beta polypeptide 2 (neuronal); Polymorphism: rs4845378); CLU (Clusterin, Polymorphism: rs11136000), also known as apolipoprotein J (apoJ); CR1 (Complement component (3b/4b) receptor 1; Polymorphism: rs6656401); CST3 (Cystatin c; Polymorphism: rs1064039); hCG2039140 (Polymorphism: rs1903908); IL1B (Interleukin 1, beta; Polymorphism: rs1143634); PICALM (Phosphatidylinositol-binding clathrin assembly protein; Polymorphism: rs541458); SORCS1 (Sortilin-related vpsl0 domain containing receptor 1; Polymorphism: rs600879); SORL1 (Sortilin-related receptor; Polymorphism: rs12285364); TFAM (Transcription factor a, mitochondrial; Polymorphism: rs2306604); TNK1 (Tyrosine kinase, non-receptor, 1; Polymorphism: rs1554948). Mutations in the amyloid precursor protein (APP) gene and presenilin-1 (PSEN1) and presenilin-2 (PSEN2) genes are also associated with increased risk of Alzheimer's disease.

### 4. DESCRIPTION OF THE DRAWINGS

FIG. 1A-B: Systemic administration of TMP attenuates neuronal degeneration in rat brains following kainate-induced seizures. One month-old Sprague-Dawley rats were subcutaneously (s.c.) administrated kainite (KA) (10.5 mg/Kg). 1 hr after the onset of seizures animals were given TMP (50 mg/Kg, s.c.) or equal volume of vehicle. All the animals were sacrificed 24 hr after TMP or vehicle injection under deep anesthesia. (A) Both hematoxylin-eosin (H&E) and TUNEL staining demonstrate massive neuronal damage shown as pink in H&E staining (f, g) and green in TUNEL (h, i & j) in both hippocampal (f, g, h & i) and piriform cortex (j) regions. In contrast, the animals received TMP only exhibited a few sporadic pink (1) and TUNEL-positive cells in the corresponding regions (m, n & o). Quantification of TUNEL-positive cells according to our previous methods reveals a significant less number of damaged neurons in the hippocampal regions with TMP treatment. Scale bar=100 μm in b, d, e, g, i, j, 1, n and o; and 400 μm in a, c, f, h, k and m.

FIG. 2A-F: TMP promotes neuronal survival in neuronal cultures treated with Aβ1-42. Primary neurons prepared from postnatal 1 day C57BL mice were treated with pre-aggregated Aβ1-42 combined with either 50 μM TMP or equal volume of vehicle at day 5 after plating. (A-C) 24 hr after treatments differential interference contrast (DIC) microscopy reveals remarkable morphological changes showing neuronal degeneration in the cells treated with Aβ1-42 (B) but not in these cells in the presence of TMP (C). (D-F) TMRM, a mitochondrial membrane potential indictor, staining reveals mitochondrial depolarization in most Aβ1-42-treated cells (E) and very few cells in the cultures with “Aβ1-42+TMP” (F). Nuclei were counterstained as blue by Hoechst 33342 (Hoec). Scale bar=100 μm in b & e; 80 μm a, d, f; 60 μm in c; and 40 μm in B.

FIG. 3A-D: TMP attenuates Aβ-related pathology in 3×Tg-AD mouse brain. (A) 16-month old triple-transgenic mice (3×Tg-AD, females) were fed with TMP containing (300 mg TMP/Kg diet) or nutrients-matched control diet made by BioSery (Frenchtown, N.J.) for 70 days. Under this circumstance, each experiment mouse continuously received TMP per os at a daily dosage of about 40˜50 mg/Kg (weight) based on the observations of the body weight versus the daily diet consumption during the experimentation period. As previously reported, no adverse effects on the health of animals have been observed. Animals were euthanized and perfused with PBS following behavioral analyses. Half the brain was fixed with 4% paraformaldehyde (PFA) in 1×PBS and sectioned using a microtome at 40 μm, while the other half-brain was dissected and rapidly frozen for biochemical analyses. (A) Immunohistochemistry using microtome sections and a specific IgG against Aβ (BAM01, NeoMarker) and either fluorescein (FITC) (A.a-c) or diaminobenzidine (DAB) (A.d,e) detection reveals the intensive staining of Aβ in both senile plaques and cells a cortical region of brain sections from the animals treated by vehicle (A a,b,d). The boxed areas in (a) is shown in (b) at higher magnification. In contrast, markedly reduced levels of Aβ staining were detected in the corresponding region of brain sections from the animals treated by TMP (A.c,e). (B) Immunofluorescence of glial fibrillary acidic protein (GFAP, green) reveals strong GFAP signal suggesting the activation of astrocytes in AD mouse brain with control diet (B.a) and reduced intensity of GFAP staining in the corresponding region from the animals received TMP (B.c). The boxed areas in a & c are shown in b & d at higher magnification. Nuclei were stained as red by propidium iodide (PI). Scale bar=400 μm in A.a&c; 100 μm in B.a&c; 80 μm A.d&e, B.d; and 60 μm in A.b, B.b. (C) Glutathione (GSH) contents in the mouse brains were measured in the cortical lysates prepared from the 3×Tg AD mouse brains following TMP treatments or control using a Biovison Glutathione colorimetric detection kit. Lysates from age-matched untreated wild type mouse brains were used as controls (WT). The results suggest that TMP significantly increase GSH contents in Alzheimer's mouse brains. (D) To exclude a potential possibility that TMP-mediated changes of Aβ and tau expression in 3×Tg-AD mouse brain may result from the direct suppression of TMP on Thy1.2 promoter, which directs expression of all three transgenes in 3×Tg-AD mouse, a luciferase-mediated Thy1.2 activity reporter (kindly provided by Dr. Green & LaFerla) were co-transfected with DsRed2-mito vector into mouse neuroblastoma N2A cells, treated with TMP (50 μM) or vehicle, and stained with a luciferase assay kit 24 hr after. Quantification of fluorescein (luciferase)-positive cells relative to all transfected cells based on DsRed2 fluorescence (RFP) reveals no influence of TMP on Thy1.2 activity.

FIG. 4A-D: TMP improves learning and memory in 3×Tg AD mice. Both 16-month old 3×Tg-AD and background-matched wild type (WT) mice (females) were fed with TMPcontaining or nutrients matched control for 60 days as described in FIG. 3. (A) On day 61, all the mice were tested for object recognition as previously described. Briefly, mice (n=14 for each group) were habituated to a 60×30 cm container for 5 min. Two identical objects were placed in the bottom of the cage as mirror images of each other. The animal was trained for 5 min followed by a 5 min retention interval in the home cage. Then one of the objects in the test container was replaced with a novel object. During the 3-min probe trial, the mouse was placed in the cage and time spent investigating both objects was measured. A recognition index (R1) score is depicted as: (TimeN (in seconds for novel object)/TimeTotal (in seconds for both old and novel objects))×100. (B-D) Starting on day 62, all the mice were trained for 9 days on the Morris water maze (MWM) exactly as described for the behavior testing. MWM acquisition demonstrates that TMP treatment significantly improved the performance of 3×Tg-AD mice (B). (C) MWMprobe trial for number of platform location crosses, and (D) MWM probe trial for time spent in the target quadrant. As shown by the p values, (*) indicates significant improvement by TMP treatments with respect to the corresponding controls.

FIG. 5A-D: TMP inhibits production of peroxides in cells under stress.

FIG. 6: TMP reduces Aβ plaques in the retinas of 3×Tg-AD mice.

FIG. 7: TMP reduces both fibril and oligomer conformations of Aβ in 3×Tg-AD mice.

FIG. 8: TMP significantly reduces the number if Aβ plaques within 3×Tg-AD mouse retinas.

FIG. 9: Astrocytic activation in the retinas of 3×Tg-AD mice is inhibited by TMP.

FIG. 10: TMP reduces BACE-related APP cleavage and Aβ accumulation in 3×Tg AD mouse brains.

FIG. 11: APP accumulates in the retina of 3×Tg-AD mice.

FIG. 12: Aβ plaques are found in the photoreceptor layer of 3×Tg-AD mice.

FIG. 13: Aβ plaques in the optic nerve of 3×Tg-AD mice.

FIG. 14A-R: Accumulation of senile plaques in Tg2576 Alzheimer's mouse retinas is altered by Aβ-based immunotherapy. Immunofluorescence microscopy following BAM01 antibody staining reveals no signal in the wild-type (A) and robust Aβ senile plaques in Tg2576 mouse brain (B). DAB staining following APP antibody labeling in cross-retinal sections shows endogenous background signal (light brown) and expression of transgene APPsw product (dark brown) in wild-type (C) and Tg2576 (D) mice, respectively. E-H: BAM01 for Aβ yields low background signal in the wild-type (E) and plaque-like Aβ deposits in Tg2576 (F, arrow) mouse retinas; a different field at higher magnification for the control (G) and Tg2576 (H, arrows indicate plaque-like Aβ deposits) mouse retinas. The adjacent section to F was stained with 6E10 antibody and visualized with immunofluorescence microscopy (I). The boxed area in I is shown in J at a higher magnification. Sections close to J were stained with a monoclonal antibody for Aβ₄₀ (K) or for Aβ₄₂ (L) or with Congo red (M). A section from a different Tg2576 mouse stained with Congo red demonstrates condensed Aβ plaques that are similar to those detected in AD brain and mouse retinas by others (P, arrow) (Perez et al., 2009). N: The adjacent section to I was stained with AT8 for hyperphosphorylated tau and visualized by microscopy following DAB staining The boxed area in N is shown in 0 at a higher magnification (arrow indicates plaque-like staining) Cell nuclei were counterstained purple by hematoxylin (C—H, N, and O) or blue by 4′,6′-diamidino-2-phenylindole (K and L). The pigment epithelium-choroid layer (p) is dark brown or black in DAB-stained sections (E, F, and N) and provides red background fluorescence with sclera (s). Quantification of Aβ staining for the cross retinal sections from all of the animals shows percentage of Aβ-positive sections (O) and number of Aβ plaques per section (R). WT, wild-type; Tg, transgenic; Ctl, Control (sham-vaccinated Tg2576); AF, Aβ fibrils. Bars depict mean±SEM (n=7-9), *P<0.05. os, photoreceptor outer segments; onl, outer nuclear layer; opl, outer plexiform layer; inl, inner nuclear layer; ipl, inner plexiform layer; g, ganglion cell layer. Scale bar=800 μm (A and B), 60 μm (C, D, G, H, J, K, L, M, O, and P), and 300 gm (E, F, I, and N).

FIG. 15A-W: Capillary deposition of Aβ in the retinas from Tg2576 mice following vaccinations. Immunoreactivity of Aβ (red) and capillary the endothelial cell marker vWF (green) was visualized in Tg2576 (Tg, G-R) and wild-type (WT, A-F) mice, with or without AO immunization, by immunofluorescence microscopy. Boxed areas in C, I, and O are shown in F, L, and R, respectively, at a higher magnification. Regions labeled s and p in G-I and M-O denote background fluorescence from sclera and pigment epithelium-choroid layer, respectively. S-V: A field in a cross-retinal section from an AO-immunized Tg2576 mouse shows that Aβ immunoreactivity (red in S and U) overlaps with vWF immunoreactivity within microvasculature (green in T, arrowheads) or surrounds vWF staining in the wall of a microvessel (green in V, arrow). inl, inner nuclear layer; ipl, inner plexiform layer; g, ganglion cell layer. W: Quantification of angiopathy scores for each group of animals. Bars depict mean±SEM (n=7-9); *P<0.05; **P<0.005. WT, wild-type; Tg, transgenic; Ctl, Control (sham-vaccinated Tg2576); AF, Aβ fibrils. Scale bar=300 μm (A-C, G-I, and M-O), 60 μm (D-F, J-L, and P-R), and 20 μm (S-V).

FIG. 16A-Q: Microglial infiltration is associated with Aβ deposition in the retinas from Tg2576 mice following vaccinations. Immunofluorescence microscopy reveals IBA1 immunoreactivity as microglial marker (green) and Aβ deposition (red) in the retinas from different groups of wild-type (A-C) or Tg2576 mice (D-O) as indicated at the top of the figure. P: A field shows both IBA1 (green) and Aβ immunoreactivity in a cross-retinal section from an “AF”-immunized Tg2576 mouse at a higher magnification. Q: Quantification of IBA1-immunoreactive cells within a retina. Bars depict mean±SEM (n=7-9); *P<0.05, **P<0.001. WT, wild-type; Tg, transgenic; Ctl, Control (sham-vaccinated Tg2576); AF, Aβ fibrils. os, photoreceptor outer segments; onl, outer nuclear layer; opl, outer plexiform layer; inl, inner nuclear layer; ipl, inner plexiform layer; g, ganglion cell layer. Scale bar=160 μm (A-O) and 20 μm (P).

FIG. 17: Enhanced astrogliosis in Tg2576 mouse retinas following vaccinations. Immunofluorescence microscopy reveals GFAP immunoreactivity (green) as a marker for astrocytes or Müller cells and Aβ deposition (red) in the retinas from different treatment groups as indicated at the top of the figure. Cell nuclei are counterstained blue by 4′,6′-diamidino-2-phenylindole in the merged images. Background fluorescence is evident in the sclera (s) and pigment epithelium-choroid layer (p) in columns AO and AF. WT, wild-type; Tg, transgenic; Ctl, Control (sham-vaccinated Tg2576); AF, Aβ fibrils; onl, outer nuclear layer; opl, outer plexiform layer; inl, inner nuclear layer; ipl, inner plexiform layer; g, ganglion cell layer. Scale bar=90 μm.

FIG. 18: Morphormetric analysis of the retinas from wild-type mice and Tg2576 mice that received different vaccinations. Measurements of retinal thickness were made from the ganglion cell layer to the outside border of the outer nuclear layer. WT, wild type; Tg, transgenic; Ctl, Control (sham-vaccinated Tg2576); AF, Aβ fibrils. Bars depict mean±SEM (n=7-9).

FIG. 19A-D: Accumulation of Aβ deposits in Tg2576 Alzheimer's mouse brain is altered by Aβ-based immunotherapy. Coronal sections through the hippocampus were incubated with 6E10 antibody and visualized by diaminobenzidine staining Light microscopy demonstrated robust Aβ senile plaques in both hippocampal and neocortical areas in Tg2576 mouse brain (A) and also shown in FIG. 14B by fluorescence microscopy. In contrast, notably less Aβ deposits were detected in the corresponding regions in Tg2576 mice following vaccination with Aβ fibrils (AF, B), Aβ oligomers (AO, C) or the islet amyloid polypeptide (IAPP, D).

FIG. 20: GC-MS analysis of two differently-sourced TMP preparations. Both synthetic and herb-extracted TMP preparations were analyzed by GC-MS through the Mass Spectrometry Facility at UC Irvine. The results reveal that the two differently sourced TMP preparations were ultra-pure (>99.9%).

FIG. 21A-D: Improvement of learning and memory in aged 3×Tg AD mice by TMP treatment. Novel object recognition task was performed. A recognition index (R1) score is depicted as: [Time_{N} (in seconds for novel object)/Time_{Total} (in seconds for both old and novel objects)]×100. B-D Starting on day 62, all the mice were trained for 9 days on the Morris water maze (MWM) for the behavior testing. MWM acquisition demonstrated that the treatment significantly improved the performance of 3×Tg-AD mice (B). (C) MWM probe trial for number of platform location crosses, and (D) MWM probe trial for time spent in the target quadrant. As shown by the p values, (*) indicates significant improvement by the compound treatments with respect to the corresponding controls.

FIG. 22: Improvement of memory in 5×FAD mice by synthetic form of TMP treatments. Novel object recognition task was performed and depicted as FIG. 21. While TMP-N demonstrated some slight negative effect on the memory performance for both wild type and 5×FAD demented mice, TMP-S significantly improved this performance (p<0.05, N=8-12).

FIG. 23: Abeta deposition in 5×FAD mouse brains treated with TMP-N or TMP-S (see Section 6.3.1) or control (Vehicle). DAPI indicates DNA-binding DAPI stain.

FIG. 24: Preservation of retinal ganglion cells (RGC) in 5×FAD mice by TMP (see Section 6.3.1). Numbers of RGC of wild-type, control (Vehicle) and TMP-treated mice were quantified. TMP-N demonstrated slight protective effect on RGC in 5×FAD mice and TMP-S significantly increased number of RGC in the treated 5×FAD mice (*p<0.05, N=8-12 for 5×FAD, 3 for WT).

### 5. DETAILED DESCRIPTION

Presented herein are methods of treating degenerative disorders of the central nervous system using TMP.

Any degenerative disorder of the CNS can be treated in accordance with the methods provided herein including, but not limited to, AD, PD, HD, TBI, MCI, glaucoma, age-related macular degeneration, epilepsy, retinal diseases associated with Alzheimer's disease and other retinal diseases, demyelinating diseases of the CNS, multiple sclerosis (MS), transverse myelitis, optic neuritis, Devic's disease, demyelinating diseases of the peripheral nervous system, Guillain-Barré syndrome, chronic inflammatory demyelinating polyneuropathy, hemiplegia, cerebral palsy, paraplegia, quadraplegia, vascular dementia, cerebral amyloid deposits, bacterial and viral meningitis, cerebral toxoplasmosis, brain cancers (e.g., metastatic carcinoma of the brain, glioblastoma, astrocytoma, and acoustic neuroma), hydrocephalus, and encephalitis.

In one embodiment, provided herein is a method for treating a degenerative disorder of the CNS in a patient in need thereof comprising administering a therapeutically effective amount of TMP. In another embodiment, provided herein is a method for treating AD in a patient in need thereof comprising administering a therapeutically effective amount of TMP. In another embodiment, provided herein is a method of treating MCI in a patient in need thereof comprising administering a therapeutically effective amount of TMP. In another embodiment, provided herein is a method of treating PD in a patient in need thereof comprising administering a therapeutically effective amount of TMP. In another embodiment, provided herein is a method of treating HD in a patient in need thereof comprising administering a therapeutically effective amount of TMP. In yet another embodiment, presented herein is a method treating TBI in a patient in need thereof comprising administering a therapeutically effective amount of TMP.

In a specific embodiment, presented herein is a method for preventing the onset of AD in a patient at risk of developing AD, comprising administering a therapeutically effective amount of TMP to the patient at risk of developing AD. In another specific embodiment, presented herein is a method for treating early onset AD comprising administering a therapeutically effective amount of TMP to a patient with early onset AD. In another specific embodiment, presented herein is a method for treating familial AD comprising administering a therapeutically effective amount of TMP to a patient with familial AD. In another specific embodiment, presented herein is a method for treating mild AD comprising administering a therapeutically effective amount of TMP to a patient with mild AD. In another specific embodiment, presented herein is a method for treating MCI associated with AD comprising administering a therapeutically effective amount of TMP to a patient with MCI associated with AD. In another specific embodiment, presented herein is a method for treating moderate AD comprising administering a therapeutically effective amount of TMP to a patient with moderate AD. In another specific embodiment, presented herein is a method for treating severe AD comprising administering a therapeutically effective amount of TMP to a patient with severe AD. In another specific embodiment, presented herein is a method for treating late-stage AD comprising administering a therapeutically effective amount of TMP to a patient with late-stage AD. In one aspect of these embodiments, TMP is administered to the patient at a dose of 300 mg/kg/day, orally. In another aspect of these embodiments, TMP is administered to the patient at a dose of 50 mg/kg/day, s.c.

In one aspect, the methods for treating a degenerative disease of the CNS involve the administration of TMP as a single-agent therapy, to a patient in need thereof. In a specific embodiment, presented herein is a method for a degenerative disease of the CNS comprising administering to a patient in need thereof a therapeutically effective amount of TMP as a single agent. In another embodiment, presented herein is a method for treating a degenerative disease of the CNS comprising administering to a patient in need thereof a pharmaceutical composition comprising TMP, as the single active ingredient, and a pharmaceutically acceptable carrier, excipient or vehicle.

In another aspect, the methods for treating a degenerative disease of the CNS involve the administration of TMP in combination with another therapy (e.g., one or more additional therapies that do not comprise TMP) to a patient in need thereof. Such methods may involve administering TMP prior to, concurrent with, or subsequent to administration of the additional therapy. In certain embodiments, such methods have an additive or synergistic effect. In a specific embodiment, presented herein is a method for treating a degenerative disease of the CNS comprising administering to a patient in need thereof a therapeutically effective amount of TMP and a therapeutically effective amount of another therapy. In another embodiment, presented herein is a method for treating a degenerative disease of the CNS comprising administering to a patient in need thereof a pharmaceutical composition comprising TMP, as the single active ingredient, and a pharmaceutically acceptable carrier, excipient or vehicle, and a therapeutically effective amount of another therapy.

In a specific embodiment, presented herein is a method for treating a degenerative disease of the CNS comprising: (a) administering to a patient in need thereof one or more doses of TMP or a pharmaceutical composition thereof; and (b) monitoring in the patient one or more of the following pathologies: the accumulation of protein aggregates (e.g., amyloid plaques or neurofibrillary tangles (NFT)); inflammatory events; cerebral amyloid angiopathy; oxidative stress; mitochondrial stress; endoplasmic reticulum stress; disruption of axonal transport; loss of spines and/or synapses; cholinergic dysfunction; neuritic fragmentation; loss of estrogen; loss of neurotrophic factors; and/or loss of neurons; wherein treatment is determined to be effective if the reduction and/or inhibition of one or more of the pathologies is observed. In certain embodiments, the monitoring step (b) is carried out before and/or after a certain number of doses (e.g., 1, 2, 4, 6, 8, 10, 12, 14, 15, or 20 doses, or more doses; or 2 to 4, 2 to 8, 2 to 20 or 2 to 30 doses) or a certain time period (e.g., 1, 2, 3, 4, 5, 6, or 7 days; or 1, 2, 3, 4, 5, 10, 15, 20, 30, 40, 45, 48, or 50 weeks) of administering TMP or a pharmaceutical composition thereof. In certain embodiments, one or more of these monitoring parameters are detected prior to administration of TMP or pharmaceutical composition thereof. In some embodiments, a change in one or more of the monitoring parameters of step (b) following administration of TMP or pharmaceutical composition thereof may indicate that the dosage, frequency and/or length of administration of TMP or a pharmaceutical composition thereof may be adjusted (e.g., increased, reduced or maintained).

In specific embodiments, the methods for treating degenerative disease of the CNS provided herein alleviate or manage one, two or more symptoms associated with the degenerative disease of the CNS. Alleviating or managing one, two or more symptoms of a degenerative disease of the CNS may be used as a clinical endpoint for efficacy of TMP for treating the degenerative disease of the CNS. In some embodiments, the methods for treating a degenerative disease of the CNS provided herein reduce the duration and/or severity of one or more symptoms associated with the degenerative disease of the CNS. In some embodiments, the methods for treating a degenerative disease of the CNS provided herein inhibit the onset, progression and/or recurrence of one or more symptoms associated with the degenerative disease of the CNS. In some embodiments, the methods for treating a degenerative disease of the CNS provided herein reduce the number of symptoms associated with the degenerative disease of the CNS.

In particular embodiments, the methods for treating a degenerative disease of the CNS provided herein reduce the mortality of subjects diagnosed with the degenerative disease of the CNS. In other embodiments, the methods for treating a degenerative disease of the CNS provided herein prevent the development, onset or progression of a degenerative disease of the CNS, or one or more symptoms associated therewith. In particular embodiments, the methods for treating a degenerative disease of the CNS provided herein increase symptom-free survival of patients with a degenerative disease of the CNS. In some embodiments, the methods for treating a degenerative disease of the CNS provided herein do not cure the degenerative disease of the CNS in patients, but prevent the progression or worsening of the disease.

In certain embodiments, the severity and progression degenerative disorders of the CNS, e.g., AD, is monitored by measuring the retinal deposition of Aβ plaques, e.g., retinal plaques. In a specific embodiment, the severity and progression of AD is monitored by measuring the retinal deposition of Aβ plaques, e.g., retinal plaques. In another specific embodiment, the severity and progression of AD-related blindness is monitored by measuring the retinal deposition of Aβ plaques, e.g., retinal plaques. In accordance with such embodiments, the severity and/or progression of the disease/disorder is reduced if the amount of retinal deposition of Aβ plaques, e.g., retinal plaques, is reduced.

5.1 TMP

The chemical structure of TMP is presented below:

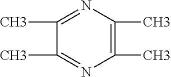

Those skilled in the art will understand that the methods presented herein encompass the use of TMP derivatives and variants that do not alter the effect of TMP. TMP and such derivatives and/or variants are collectively referred to herein as “TMP compounds.”

TMP can be provided in a pharmaceutically acceptable form, including where the compound can be formulated in the pharmaceutical compositions per se, or in the form of a hydrate, solvate, N-oxide, or pharmaceutically acceptable salt. Typically, such salts are more soluble in aqueous solutions than the corresponding free acids and bases, but salts having lower solubility than the corresponding free acids and bases can also be formed. It is to be understood that reference to the compound in discussions of formulations is also intended to include, where appropriate as known to those of skill in the art, formulation of the prodrugs of the compounds.

In one embodiment, TMP compounds are provided as non-toxic pharmaceutically acceptable salts, as noted previously. Suitable pharmaceutically acceptable salts of the compounds described herein include acid addition salts such as those formed with hydrochloric acid, fumaric acid, p-toluenesulphonic acid, maleic acid, succinic acid, acetic acid, citric acid, tartaric acid, carbonic acid, or phosphoric acid. Salts of amine groups can also include quaternary ammonium salts in which the amino nitrogen atom carries a suitable organic group such as an alkyl, alkenyl, alkynyl, or substituted alkyl moiety. Furthermore, where the compounds of the invention carry an acidic moiety, suitable pharmaceutically acceptable salts thereof can include metal salts such as alkali metal salts, e.g., sodium or potassium salts; and alkaline earth metal salts, e.g., calcium or magnesium salts.

5.2 Formulations

TMP can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient selected from fillers or diluents, binders, disintegrants, lubricants, flavoring agents, preservatives, stabilizers, suspending agents, dispersing agents, surfactants, antioxidants or solubilizers.

Excipients that may be selected are known to those skilled in the art and include, but are not limited to fillers or diluents (e.g., sucrose, starch, mannitol, sorbitol, lactose, glucose, cellulose, talc, calcium phosphate or calcium carbonate and the like), a binder (e.g., cellulose, carboxymethylcellulose, methylcellulose, hydroxymethylcellulose, hydroxypropylmethylcellulose, polypropylpyrrolidone, polyvinylpyrrolidone, gelatin, gum arabic, polyethyleneglycol or starch and the like), a disintegrant (e.g., sodium starch glycolate, croscarmellose sodium and the like), a lubricant (e.g., magnesium stearate, light anhydrous silicic acid, talc or sodium lauryl sulfate and the like), a flavoring agent (e.g., citric acid, or menthol and the like), a preservative (e.g., sodium benzoate, sodium bisulfite, methylparaben or propylparaben and the like), a stabilizer (e.g., citric acid, sodium citrate or acetic acid and the like), a suspending agent (e.g., methylcellulose, polyvinyl pyrrolidone or aluminum stearate and the like), a dispersing agent (e.g., hydroxypropylmethylcellulose and the like), surfactants (e.g., sodium lauryl sulfate, polaxamer, polysorbates and the like), antioxidants (e.g., ethylene diamine tetraacetic acid (EDTA), butylated hydroxyl toluene (BHT) and the like) and solubilizers (e.g., polyethylene glycols, SOLUTOL®, GELUCIRE® and the like). The effective amount TMP in the pharmaceutical composition may be at a level that will exercise the desired effect.

The dose of TMP to be administered to a patient is rather widely variable and can be subject to the judgment of a health-care practitioner. In general, TMP can be administered one to four times a day. The dosage may be properly varied depending on the age, body weight and medical condition of the patient and the type of administration. In one embodiment, one dose is given per day. In any given case, the amount of TMP administered will depend on such factors as the solubility of the active component, the formulation used and the route of administration.

TMP can be administered orally, with or without food or liquid.

TMP can also be administered intradermally, intramuscularly, intraperitoneally, percutaneously, intravenously, intraocularly, subcutaneously, intranasally, epidurally, sublingually, intracerebrally, intravaginally, transdermally, rectally, mucosally, by inhalation, or topically to the ears, nose, eyes, or skin (e.g, as a topical ophthalmic). The mode of administration is left to the discretion of the health-care practitioner, and can depend in-part upon the site of the medical condition.

In one embodiment, TMP is administered orally using a capsule dosage form composition, wherein the capsule contains TMP without an additional carrier, excipient or vehicle.

In another embodiment, provided herein are compositions comprising an effective amount of TMP and a pharmaceutically acceptable carrier or vehicle, wherein a pharmaceutically acceptable carrier or vehicle can comprise one or more excipients, or a mixture thereof. In one embodiment, the composition is a pharmaceutical composition.

Compositions can be formulated to contain a daily dose, or a convenient fraction of a daily dose, in a dosage unit. In general, the composition is prepared according to known methods in pharmaceutical chemistry. Capsules can be prepared by mixing TMP with one or more suitable carriers or excipients and filling the proper amount of the mixture in capsules.

The compositions provided herein can include a variety of additives including antioxidants, antimicrobial agents, enzyme inhibitors, stabilizers, preservatives, flavors, sweeteners and further components known to those skilled in the art.

In certain embodiments, TMP is purified. In specific embodiments, TMP is at least 75% pure, at least 80% pure, at least 85% pure, at least 90% pure, at least 95% pure, at least 96% pure, at least 97% pure, at least 98% pure, at least 99% pure, at least 99.5% pure, at least 99.9% pure, or at least 100% pure.

5.3 Dosage and Administration

In accordance with the methods for treating a degenerative disorder of the CNS provided herein, TMP or a pharmaceutical composition thereof can be administered to a subject in need thereof by a variety of routes in amounts which result in a beneficial or therapeutic effect. TMP or pharmaceutical composition thereof may be orally administered to a subject in need thereof in accordance with the methods for treating a degenerative disorder of the CNS provided herein. The oral administration of TMP or a pharmaceutical composition thereof may facilitate subjects in need of such treatment complying with a regimen for taking TMP or pharmaceutical composition. Thus, in a specific embodiment, TMP or pharmaceutical composition thereof is administered orally to a subject in need thereof.

Other routes of administration include, but are not limited to, intravenous, intradermal, intramuscular, subcutaneous, intranasal, inhalation, transdermal, topical, transmucosal, intracranial, intrathecal, intraocular, intraurethral, epidural and intra-synovial. In one embodiment, TMP or a pharmaceutical composition thereof is administered systemically (e.g., parenterally) to a subject in need thereof. In another embodiment, TMP or a pharmaceutical composition thereof is administered as a topical opthalmic to a subject in need thereof. In another embodiment, TMP or a pharmaceutical composition thereof is administered locally to a subject in need thereof. In one embodiment, TMP or a pharmaceutical composition thereof is administered via a route that permits TMP to cross the blood-brain barrier (e.g., orally).

In accordance with the methods for treating a degenerative disorder of the CNS provided herein that involve administration of TMP in combination with one or more additional therapies, TMP and one or more additional therapies may be administered by the same route or a different route of administration.

The dosage and frequency of administration of TMP or a pharmaceutical composition thereof is administered to a subject in need thereof in accordance with the methods for treating a degenerative disorder of the CNS provided herein will be efficacious while minimizing any side effects. The exact dosage and frequency of administration of TMP or a pharmaceutical composition thereof can be determined by a practitioner, in light of factors related to the subject that requires treatment. Factors which may be taken into account include the severity of the disease state, general health of the subject, age, and weight of the subject, diet, time and frequency of administration, combination(s) with other therapeutic agents or drugs, reaction sensitivities, and tolerance/response to therapy. The dosage and frequency of administration of TMP or a pharmaceutical composition thereof may be adjusted over time to provide sufficient levels of TMP or to maintain the desired effect.

In certain embodiments, TMP or pharmaceutical composition thereof is administered to a subject in accordance with the methods for treating a degenerative disorder of the CNS presented herein once a day, twice a day, three times a day, or four times a day. In some embodiments, TMP or pharmaceutical composition thereof is administered to a subject in accordance with the methods for treating a degenerative disorder of the CNS presented herein once, twice, three times, or four times every other day (i.e., on alternate days); once, twice, three times, or four times every two days; once, twice, three times, or four times every three days; once, twice, three times, or four times every four days; once, twice, three times, or four times every 5 days; once, twice, three times, or four times every week, once, twice, three times, or four times every two weeks; once, twice, three times, or four times every three weeks; once, twice, three times, or four times every four weeks; once, twice, three times, or four times every 5 weeks; once, twice, three times, or four times every 6 weeks; once, twice, three times, or four times every 7 weeks; or once, twice, three times, or four times every 8 weeks. In particular embodiments, TMP or pharmaceutical composition thereof is administered to a subject in accordance with the methods for treating a degenerative disorder of the CNS presented herein in cycles, wherein TMP or a pharmaceutical composition thereof is administered for a period of time, followed by a period of rest (i.e., TMP or pharmaceutical composition is not administered for a period of time). In specific embodiments, a method for treating a degenerative disorder of the CNS presented herein involves the administration of TMP or a pharmaceutical composition thereof in cycles, e.g., 1 week cycles, 2 week cycles, 3 week cycles, 4 week cycles, 5 week cycles, 6 week cycles, 8 week cycles, 9 week cycles, 10 week cycles, 11 week cycles, or 12 week cycles. In such cycles, TMP or a pharmaceutical composition thereof may be administered once, twice, three times, or four times daily. In particular embodiments, a method for treating a degenerative disorder of the CNS presented herein involves the administration of TMP or a pharmaceutical composition thereof twice daily in 4 week cycles.

In one aspect, a method for treating a degenerative disorder of the CNS presented herein involves the administration of a unit dosage of TMP or a pharmaceutical composition thereof. The dosage may be administered as often as determined effective (e.g., once, twice or three times per day, every other day, once or twice per week, biweekly or monthly). In certain embodiments, a method for treating a degenerative disorder of the CNS presented herein involves the administration to a subject in need thereof of a unit dose of TMP or a pharmaceutical composition thereof that ranges from about 0.1 milligram (mg) to about 1000 mg, from about 1 mg to about 1000 mg, from about 5 mg to about 1000 mg, from about 10 mg to about 500 mg, from about 100 mg to about 500 mg, from about 150 mg to about 500 mg, from about 150 mg to about 1000 mg, from about 250 mg to about 1000 mg, from about 300 mg to about 1000 mg, or from about 500 mg to about 1000 mg, or any range in between. In some embodiments, a method for treating a degenerative disorder of the CNS presented herein involves the administration to a subject in need thereof of a unit dose of TMP or a pharmaceutical composition thereof of about 15 mg, 16, mg, 17 mg, 18 mg, 19 mg, 20 mg, 21, mg, 22 mg, 23 mg, 24 mg, 25 mg, 26 mg, 27 mg, 28 mg, 29 mg, 30 mg or 40 mg. In certain embodiments, a method for treating a degenerative disorder of the CNS presented herein involves the administration to a subject in need thereof of a unit dose of TMP or a pharmaceutical composition thereof of about 50 mg, 60 mg, 70 mg, 80 mg, 90 mg, 100 mg, 110 mg, 120 mg, 125 mg, 130 mg, 140 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, or 900 mg.

In some embodiments, a method for treating a degenerative disorder of the CNS presented herein involves the administration to a subject in need thereof of a unit dose of TMP or a pharmaceutical composition thereof of at least about 0.1 mg, 1 mg, 5 mg, 10 mg, 20 mg, 30 mg, 40 mg, 50 mg, 60 mg, 70 mg, 80 mg, 90 mg, 100 mg, 110 mg, 120 mg, 125 mg, 130 mg, 140 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg or more. In certain embodiments, a method for treating a degenerative disorder of the CNS presented herein involves the administration to a subject in need thereof of a unit dose of TMP or a pharmaceutical composition thereof of less than about 35 mg, less than about 40 mg, less than about 45 mg, less than about 50 mg, less than about 60 mg, less than about 70 mg, or less than about 80 mg.

In specific embodiments, a method for treating a degenerative disorder of the CNS presented herein involves the administration to a subject in need thereof of a unit dose of TMP or a pharmaceutical composition thereof of about 40 mg to about 500 mg, about 40 mg to about 200 mg, about 40 mg to about 150 mg, about 75 mg to about 500 mg, about 75 mg to about 450 mg, about 75 mg to about 400 mg, about 75 mg to about 350 mg, about 75 mg to about 300 mg, about 75 mg to about 250 mg, about 75 mg to about 200 mg, about 100 mg to about 200 mg, or any range in between. In other specific embodiments, a method for treating a degenerative disorder of the CNS presented herein involves the administration to a subject in need thereof of a unit dose of TMP or a pharmaceutical composition thereof of about 35 mg, 40 mg, 50 mg, 60 mg, 75 mg, 100 mg, 125 mg, 150 mg, 175 mg, 200 mg, 225 mg, 250 mg or 300 mg. In some embodiments, a method for treating a degenerative disorder of the CNS presented herein involves the administration to a subject in need thereof of a unit dose of TMP or a pharmaceutical composition thereof of about 350 mg, 400 mg, 500 mg, 600 mg, 700 mg, 800 mg, 900 mg, or 1000 mg. In some embodiments, a unit dose of TMP or a pharmaceutical composition thereof is administered to a subject once per day, twice per day, three times per day; once, twice or three times every other day (i.e., on alternate days); once, twice or three times every two days; once, twice or three times every three days; once, twice or three times every four days; once, twice or three times every five days; once, twice, or three times once a week, biweekly or monthly, and the dosage may be administered orally.

In certain embodiments, a method for treating a degenerative disorder of the CNS presented herein involves the oral administration to a subject in need thereof of a unit dose of TMP or a pharmaceutical composition thereof that ranges from about 50 mg to about 500 mg per day. In some embodiments, a method for treating a degenerative disorder of the CNS presented herein involves the oral administration to a subject in need thereof of a unit dose of TMP or a pharmaceutical composition thereof that ranges from about 80 mg to about 500 mg per day, about 100 mg to about 500 mg per day, about 80 mg to about 400 mg per day, about 80 mg to about 300 mg per day, about 80 mg to about 200 mg per day, about 200 mg to about 300 mg per day, about 200 mg to about 400 mg per day, about 200 mg to about 500 mg per day, or any range in between. In a specific embodiment, a method for treating a degenerative disorder of the CNS presented herein involves the oral administration to a subject in need thereof of a unit dose of about 40 mg of TMP or a pharmaceutical composition thereof twice per day. In another specific embodiment, a method for treating a degenerative disorder of the CNS presented herein involves the oral administration to a subject in need thereof of a unit dose of about 80 mg of TMP or a pharmaceutical composition thereof twice per day. In specific embodiments, a method for treating a degenerative disorder of the CNS presented herein involves the oral administration to a subject in need thereof of a unit dose of about 150 mg to about 250 mg, about 175 mg to about 250 mg, about 200 mg to about 250 mg, or about 200 mg to about 225 mg of TMP or a pharmaceutical composition thereof twice per day.

In some embodiments, a method for treating a degenerative disorder of the CNS presented herein involves the administration of a dosage of TMP or a pharmaceutical composition thereof that is expressed as mg per meter squared (mg/m²). The mg/m² for TMP may be determined, for example, by multiplying a conversion factor for an animal by an animal dose in mg per kilogram (mg/kg) to obtain the dose in mg/m² for human dose equivalent. For regulatory submissions the FDA may recommend the following conversion factors: Mouse=3, Hamster=4.1, Rat=6, Guinea Pig=7.7. (based on Freireich et al., Cancer Chemother. Rep. 50(4):219-244 (1966)). The height and weight of a human may be used to calculate a human body surface area applying Boyd's Formula of Body Surface Area. In specific embodiments, a method for treating a degenerative disorder of the CNS presented herein involves the administration to a subject in need thereof of an amount of TMP or a pharmaceutical composition thereof in the range of from about 0.1 mg/m² to about 1000 mg/m², or any range in between.

Other non-limiting exemplary doses of TMP or a pharmaceutical composition that may be used in the methods for treating a degenerative disorder of the CNS provided herein include mg amounts per kg of subject or sample weight. In certain embodiments, a method for treating a degenerative disorder of the CNS presented herein involves the administration to a subject in need thereof of a dosage of TMP or a pharmaceutical composition thereof that ranges from about 0.001 mg/kg to about 500 mg/kg, from about 0.01 mg/kg to about 500 mg/kg, from about 0.1 mg/kg to about 500 mg/kg, from about 1 mg/kg to about 500 mg/kg, from about 10 mg/kg to about 500 mg/kg, from about 100 mg to about 500 mg/kg, from about 150 mg/kg to about 500 mg/kg, from about 250 mg/kg to about 500 mg/kg, or from about 300 mg/kg to about 500 mg/kg. In some embodiments, a method for treating a degenerative disorder of the CNS presented herein involves the administration to a subject in need thereof of a dosage of TMP or a pharmaceutical composition thereof that ranges from about 0.001 mg/kg to about 100 mg/kg, from about 0.001 mg/kg to about 50 mg/kg, from about 0.001 mg/kg to about 25 mg/kg, from about 0.001 mg/kg to about 10 mg/kg, from about 0.001 mg/kg to about 5 mg/kg; from about 0.001 mg/kg to about 1 mg/kg; or from about 0.001 mg/kg to about 0.01 mg/kg. In accordance with these embodiments, the dosage may be administered once, twice or three times per day, every other day, or once or twice per week and the dosage may be administered orally.

In certain embodiments, a method for treating a degenerative disorder of the CNS presented herein involves the administration to a subject in need thereof of a dosage of TMP or a pharmaceutical composition thereof that ranges from about 0.01 mg/kg to about 500 mg/kg, from about 0.01 mg/kg to about 400 mg/kg, from about 0.01 mg/kg to about 300 mg/kg, from about 0.01 mg/kg to about 200 mg/kg, from about 0.01 mg/kg to about 100 mg/kg, from about 0.01 mg/kg to about 50 mg/kg, from about 0.01 mg/kg to about 25 mg/kg, from about 0.01 mg/kg to about 10 mg/kg, from about 0.01 mg/kg to about 5 mg/kg, from about 0.01 mg to about 1 mg/kg, or from about 0.01 mg/kg to about 0.1 mg/kg. In accordance with these embodiments, the dosage may be administered once, twice or three times per day, every other day, or once or twice per week and the dosage may be administered orally.

In specific embodiments, a method for treating a degenerative disorder of the CNS presented herein involves the oral administration to a subject in need thereof of a dosage of TMP or a pharmaceutical composition thereof of about 100 mg/kg to about 200 mg/kg, about 100 mg/kg to about 300 mg/kg, about 100 mg/kg to about 400 mg/kg, about 100 mg/kg to about 500 mg/kg, about 100 mg/kg to about 600 mg/kg, administered once per day. In a specific embodiment, 300 mg/kg of TMP is administered to a subject once per day, orally.

In specific embodiments, a method for treating a degenerative disorder of the CNS presented herein involves the subcutaneous administration to a subject in need thereof of a dosage of TMP or a pharmaceutical composition thereof of about 100 mg/kg to about 200 mg/kg, about 100 mg/kg to about 300 mg/kg, about 100 mg/kg to about 400 mg/kg, about 100 mg/kg to about 500 mg/kg, about 100 mg/kg to about 600 mg/kg once per day. In a specific embodiment, 50 mg/kg of TMP is administered to a subject once per day, subcutaneously.

In specific aspects, a method for treating a degenerative disorder of the CNS presented herein involves the administration to a subject in need thereof of TMP or a pharmaceutical composition thereof at a dosage that achieves a target plasma concentration of TMP in a subject with the degenerative disorder of the CNS or an animal model with a pre-established degenerative disorder of the CNS.

In some embodiments, a method for treating a degenerative disorder of the CNS presented herein involves the administration to a subject in need thereof of one or more doses of an effective amount of TMP or a pharmaceutical composition, wherein the effective amount may or may not be the same for each dose. In particular embodiments, a first dose of TMP or pharmaceutical composition thereof is administered to a subject in need thereof for a first period of time, and subsequently, a second dose of TMP is administered to the subject for a second period of time. The first dose may be more than the second dose, or the first dose may be less than the second dose. A third dose of TMP also may be administered to a subject in need thereof for a third period of time.

The length of time that a subject in need thereof is administered TMP or a pharmaceutical composition in accordance with the methods for treating a degenerative disorder of the CNS presented herein will be the time period that is determined to be efficacious. In certain embodiments, a method for treating a degenerative disorder of the CNS presented herein involves the administration of TMP or a pharmaceutical composition thereof for a period of time until the severity and/or number of symptoms associated with the degenerative disorder of the CNS decrease.

In some embodiments, a method for treating a degenerative disorder of the CNS presented herein involves the administration of TMP or a pharmaceutical composition thereof for up to 48 weeks. In other embodiments, a method for treating a degenerative disorder of the CNS presented herein involves the administration of TMP or a pharmaceutical composition thereof for up to 4 weeks, 8 weeks, 12 weeks, 16 week, 20 weeks, 24 weeks, 26 weeks (0.5 year), 52 weeks (1 year), 78 weeks (1.5 years), 104 weeks (2 years), or 130 weeks (2.5 years) or more.

In certain embodiments, a method for treating a degenerative disorder of the CNS presented herein involves the administration of TMP or a pharmaceutical composition thereof for an indefinite period of time. In some embodiments, a method for treating a degenerative disorder of the CNS presented herein involves the administration of TMP or a pharmaceutical composition thereof for a period of time followed by a period of rest (i.e., a period wherein TMP is not administered) before the administration of TMP or pharmaceutical composition thereof is resumed.

In certain embodiments, TMP is isolated directly from an extract of Ligusticum wallichii. In other embodiments, TMP is chemically synthesized.

In certain embodiments, TMP is administered in a crystalline form that solubilizes in the gut of the patient and subsequently enters the bloodstream after solubilization in the gut. In one aspect of these embodiments, the crystalline form of TMP is insoluble at neutral pH but soluble in acidic pH.

In other embodiments, a slow-release formulation of TMP is provided. In accordance with such embodiments, TMP is provided in a crystalline form that slowly solubilizes, thus slowly releasing TMP, upon which TMP can yield its effect. Slow-releasing formulations of TMP allow for reduced administration of TMP while retaining therapeutically effective doses of plasma and/or brain concentrations of TMP.

5.4 Patient Population

In some embodiments, TMP or a pharmaceutical composition thereof is administered to a subject suffering from a degenerative disorder of the CNS. In other embodiments, TMP or a pharmaceutical composition thereof is administered to a subject predisposed or susceptible to a degenerative disorder of the CNS. In a specific embodiment, TMP or pharmaceutical composition thereof is administered to a subject suffering from a degenerative disorder of the CNS to treat the degenerative disorder of the CNS. In another specific embodiment, TMP or a pharmaceutical composition thereof is administered to a subject predisposed or susceptible to a degenerative disorder of the CNS to prevent the degenerative disorder of the CNS.

In certain embodiments, TMP or pharmaceutical composition thereof is administered to a human that has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In some embodiments, TMP or pharmaceutical composition thereof is administered to a human infant. In other embodiments, TMP or pharmaceutical composition thereof is administered to a human toddler. In other embodiments, TMP or pharmaceutical composition thereof is administered to a human child. In other embodiments, TMP or pharmaceutical composition thereof is administered to a human adult. In yet other embodiments, TMP or pharmaceutical composition thereof is administered to an elderly human.

In some embodiments, TMP or pharmaceutical composition thereof is administered to a patient to treat the onset of a degenerative disorder of the CNS in a patient at risk of developing a degenerative disorder of the CNS. In some embodiments, TMP or pharmaceutical composition thereof is administered to a patient who is susceptible to adverse reactions to conventional therapies. In some embodiments, TMP or pharmaceutical composition thereof is administered to a patient who has proven refractory to therapies other than compounds, but are no longer on these therapies.

In some embodiments, the subject being administered TMP or a pharmaceutical composition thereof has not received therapy prior to the administration of TMP or pharmaceutical composition thereof.

In some embodiments, the subject is suffering from or predisposed to one or more of the following degenerative diseases of the CNS: AD, PD, HD, TBI, MCI, glaucoma, age-related macular degeneration, epilepsy, retinal diseases associated with Alzheimer's disease and other retinal diseases, demyelinating diseases of the CNS, multiple sclerosis (MS), transverse myelitis, optic neuritis, Devic's disease, demyelinating diseases of the peripheral nervous system, Guillain-Barré syndrome, chronic inflammatory demyelinating polyneuropathy, hemiplegia, cerebral palsy, paraplegia, quadraplegia, vascular dementia, cerebral amyloid deposits, bacterial and viral meningitis, cerebral toxoplasmosis, brain cancers (e.g., metastatic carcinoma of the brain, glioblastoma, astrocytoma, and acoustic neuroma), hydrocephalus, and encephalitis. In a specific embodiment the patient is suffering from or predisposed to AD. In another specific embodiment, the patient is suffering from or predisposed to familial AD. In a specific embodiment, the patient has early onset AD. In another specific embodiment, the patient has mild AD. In a specific embodiment, the patient has moderate AD. In a specific embodiment, the patient has moderately severe AD. In a specific embodiment, the patient has severe AD. In a specific embodiment, the patient has late-stage AD. In a specific embodiment, the patient has MCI associated with AD.

In a specific embodiment, the patient is suffering from or predisposed to PD. In another specific embodiment, the patient is suffering from or predisposed to HD. In another specific embodiment, the patient is suffering from TBI.

5.5 Combination Products

Any therapy which is known to be useful, or which has been used, will be used or is currently being used for the treatment of degenerative disorder of the CNS can be used in combination with TMP.

In specific embodiments, the interval of time between the administration of TMP and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In certain embodiments, TMP and one or more additional therapies are administered less than 1 day, 1 week, 2 weeks, 3 weeks, 4 weeks, one month, 2 months, 3 months, 6 months, 1 year, 2 years, or 5 years apart.

In some embodiments, the combination therapies provided herein involve administering TMP daily, and administering one or more additional therapies once a week, once every 2 weeks, once every 3 weeks, once every 4 weeks, once every month, once every 2 months (e.g., approximately 8 weeks), once every 3 months (e.g., approximately 12 weeks), or once every 4 months (e.g., approximately 16 weeks). In certain embodiments, TMP and one or more additional therapies are cyclically administered to a subject. Cycling therapy involves the administration of TMP for a period of time, followed by the administration of one or more additional therapies for a period of time, and repeating this sequential administration. In certain embodiments, cycling therapy may also include a period of rest where TMP or the additional therapy is not administered for a period of time (e.g., 2 days, 3 days, 4 days, 5 days, 6 days, 7 days, 1 week, 2 weeks, 3 weeks, 4 weeks, 5 weeks, 10 weeks, 20 weeks, 1 month, 2 months, 3 months, 4 months, 5 months, 6 months, 7 months, 8 months, 9 months, 10 months, 11 months, 12 months, 2 years, or 3 years). In an embodiment, the number of cycles administered is from 1 to 12 cycles, from 2 to 10 cycles, or from 2 to 8 cycles.

The combination of TMP and one or more additional therapies can be administered to a subject in the same pharmaceutical composition. Alternatively, TMP and one or more additional therapies can be administered concurrently to a subject in separate pharmaceutical compositions. TMP and one or more additional therapies can be administered sequentially to a subject in separate pharmaceutical compositions. TMP and one or more additional therapies may also be administered to a subject by the same or different routes of administration.

In specific embodiments, TMP is administered in combination with a drug approved for the treatment of AD, e.g., Aricept®, Razadyne®, Namenda®, Exelon®, and/or Cognex®.

5.6 Animal Models

TMP can be tested for biological activity using animal models for degenerative disorders of the CNS. Non-limiting examples include animals engineered to demonstrate one or more symptoms of a degenerative disorder of the CNS. Such animal model systems include, but are not limited to, rats, mice, chicken, cows, monkeys, pigs, dogs, rabbits, etc. In a specific embodiment, TMP is tested in a mouse model system.

The ability of TMP to treat a degenerative disorder of the CNS can be determined by administering TMP to an animal model and verifying that TMP is effective in reducing the severity of the degenerative disorder of the CNS in said animal model

5.7 Endpoints

The effectiveness of TMP in treating a degenerative disorder of the CNS can be determined by using various endpoints, e.g., ADAS; ADAS-COG; CDR-SB; and/or MMSE.

5.8 Kits

Described herein are kits comprising TMP, in one or more containers, and instructions for use.

5.9 Diagnostic Methods

The invention further relates to methods utilizing retinal imaging to diagnose and monitor efficacy of treatments for CNS disorders such as AD, PD, and HD, as well as TBI. In a particular embodiment, the invention relates to methods utilizing retinal imaging to diagnose and monitor efficacy of treatments for AD. In one embodiment retinal imaging is performed on a patient to determine levels of pathological changes in the retina to diagnose that the patient has a CNS disorder, such as AD, PD, and HD, in particular AD. In accordance with the present invention retinal imaging is performed on a patient prior to the onset of the therapeutic regimen and during or at the completion of the therapeutic regimen, wherein an improvement in the pathology indicates that the therapy is effective. In accordance with the present invention, determining the presence of Aβ plaques and amyloid angiopathy in the retina of a patient may be used to diagnose and monitor efficacy of therapeutic methods. A decrease in the presence of Aβ plaques in the retina indicates that the therapeutic regimen is effective. An improvement in amyloid angiopathy in the retina indicates that the therapeutic regimen is effective. In accordance with the present invention, determining improvement in retinal pathology, including a decrease in retinal thickness, indicates that the therapeutic regimen is effective.

The invention further relates to methods utilizing retinal imaging to diagnose and monitor efficacy of treating CNS disorders such as AD, PD, and HD, as well as TBI, by administrating effective amounts of TMP. In a particular embodiment, the invention relates to methods utilizing retinal imaging to diagnose and monitor efficacy of treating AD, by administrating effective amounts of TMP. In accordance with the present invention, determining the presence of Aβ plaques and amyloid angiopathy in the retina of a patient may be used to diagnose and monitor efficacy of administration of TMP. A decrease in the presence of Aβ plaques in the retina indicates that the TMP therapeutic regimen is effective. An improvement in amyloid angiopathy in the retina indicates that the TMP therapeutic regimen is effective. In accordance with the present invention, determining improvement in retinal pathology, including a decrease in retinal thickness, indicates that the TMP therapeutic regimen is effective.

In accordance with the present invention, any methods known in the art can be used for retinal imaging. By way of example, but not limitation, the methods of retinal imaging include confocal laser scanning tomography, nerve fiber layer testing or analysis (confocal laser scanning tomography with polarimetry), stereophotogrammetry, and optical coherence tomography.

Confocal Laser Scanning Tomography

The confocal laser scanning tomographic ophthalmoscope is a device that scans layers of the retina to make quantitative measurements of the surface features of the optic nerve head and fundus. Other terms for confocal laser scanning tomography include: laser scanning topography, confocal scanning laser topography, electro-optic fundus imaging and scanning laser polarimetry. Types of confocal laser scanning ophthalmoscopes include:

Heidelberg Laser Tomographic Scanner or Heidelberg Retina Tomograph (HRT) (Heidelberg Engineering, Dossenheim, Germany),

TopSS Topographic Scanning System (Laser Diagnostic Technologies, San Diego, Calif.); and the

Zeiss™ Confocal Laser Scanning Ophthalmoscope. (Zeiss Humphrey Systems, Dublin, Calif.).

### Optical Coherence Tomography

In OCT, low coherence near-infrared light is split into a probe and a reference beam. The probe beam is directed at the retina while the reference beam is sent to a moving reference mirror. The probe light beam is reflected from tissues according to their distance, thickness, and refractive index, and is then combined with the beam reflected from the moving reference mirror. When the path lengths of the two light beams coincide (known as constructive interference) this provides a measure of the depth and reflectivity of the tissue that is analogous to an ultrasound A scan at a single point. A computer then corrects for axial eye movement artifacts and constructs a two dimensional B mode image from successive longitudinal scans in the transverse direction. A map of the tissue is then generated based on the different reflective properties of its components, resulting in a real-time cross-sectional histological view of the tissue.

Stereophotogrammetry

Stereophotogrammetry, (Glaucoma-Scope (OIS, Sacramento, Calif.)) measures the dimensions of the optic disc in three-dimensional space using stereophotography. Stereophotographs are taken from two camera positions with parallel optical axes. Stereoanalysis of these photographs are used to determine the three-dimensional characteristics of the optic nerve head, and for following change of the optic nerve head over time.

### 6. EXAMPLES

### 6.1 Example 1

### TMP Attenuates Neuronal Degeneration

The following example demonstrates the ability of TMP to attenuate neural degeneration.

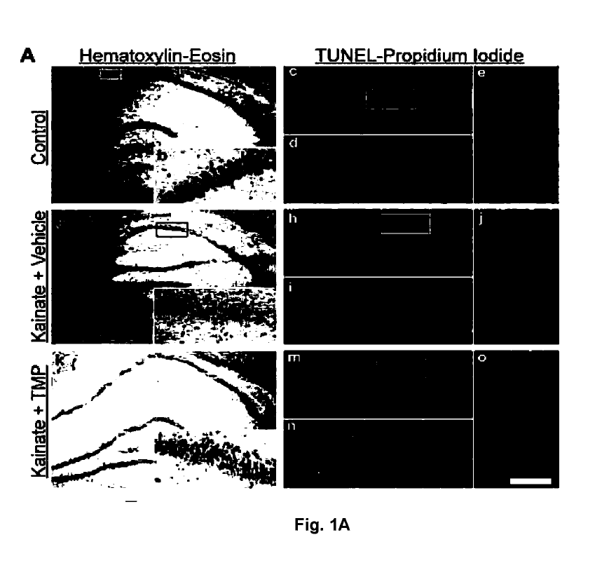

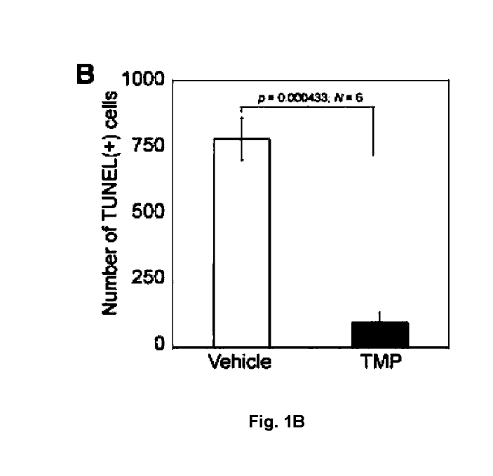

TMP attenuates neuronal degeneration in rat brains following kainite-induced seizures (See FIG. 1).

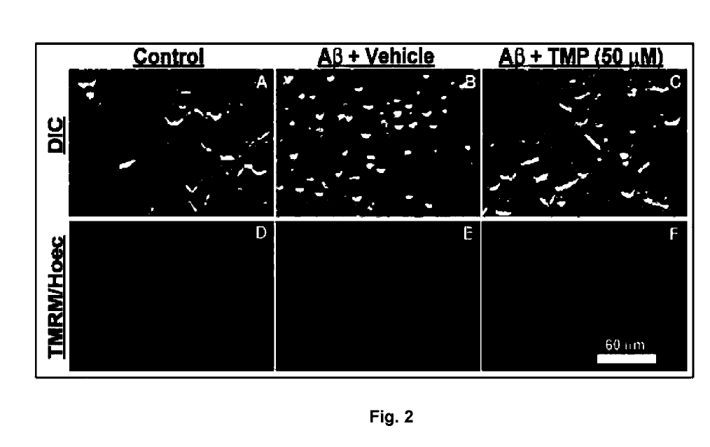

TMP promotes neuronal survival in cultures of neurons treated with Aβ₁₋₄₂ (See FIG. 2).

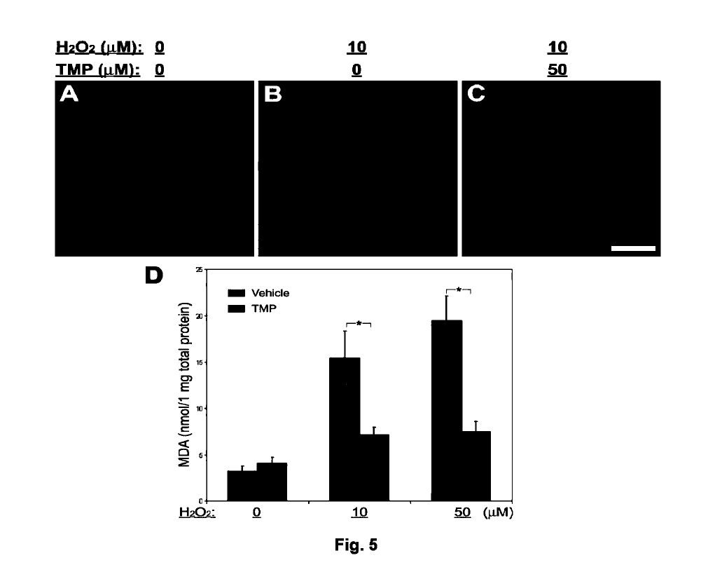

TMP inhibits the production of peroxides in cells that are under stress (See FIG. 5).

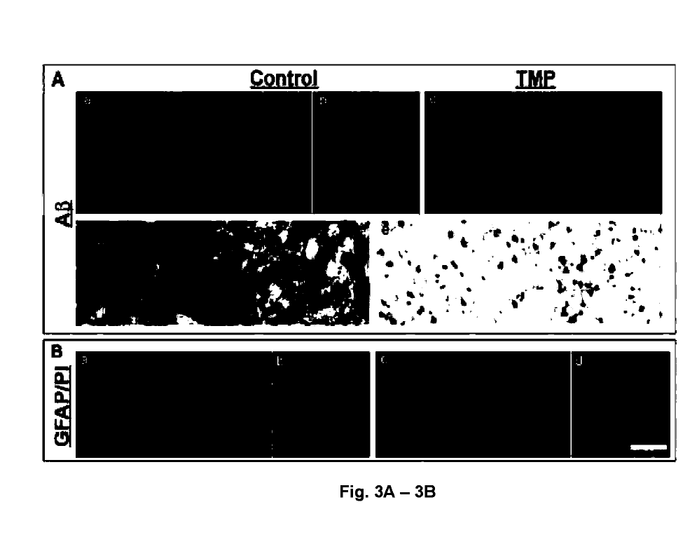

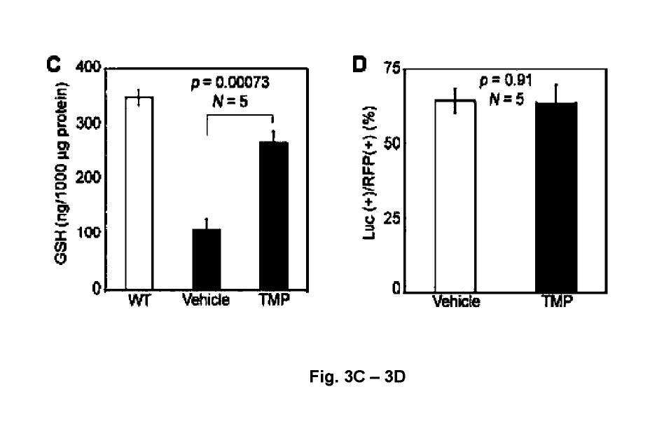

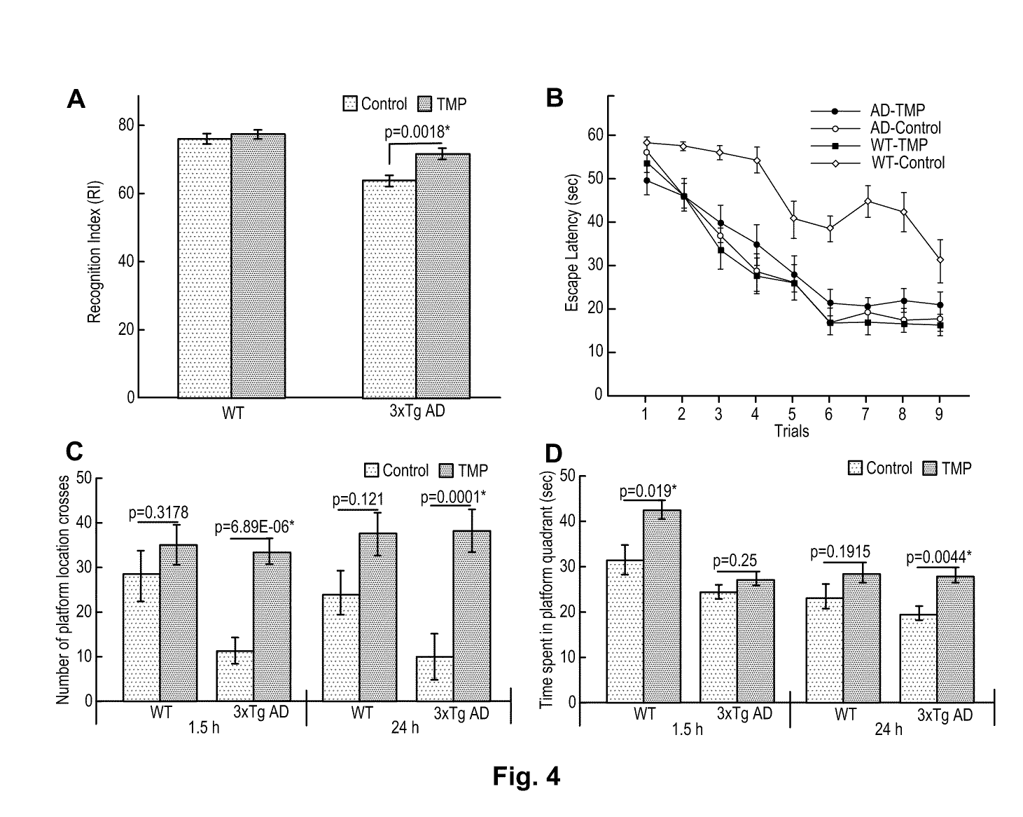

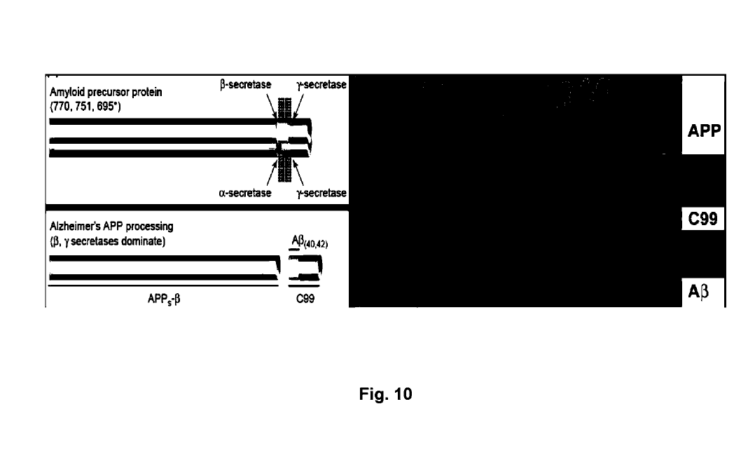

TMP improves pathology in brains of 3×Tg-AD mice and results in increased memory and learning (See FIGS. 3, 4, and 10). In these mice, the earliest pathological hallmark is the accumulation of intraneuronal Aβ in the hippocampus, cortex and amygdala in 4 month old 3×Tg-AD homozygous mice. At this age homozygous mice exhibit behavioral deficits in long term retention. Between 4-6 months of age 3×Tg-AD homozygous mice develop diffuse amyloid plaques in the neocortex. At 6 months the retention deficits are associated with significant impairments in spatial learning of the Morris Water Maze task. 16 Month old triple-transgenic female mice (3×Tg-AD) were fed TMP-containing (300 mg TMP/kg diet) or nutrients-matched control diet made by Biosery for 70 days. Brain and retina pathology was evaluated by immunohistochemistry with specific antibodies and learning and memory performance were scored using the Novel Object Recognition Task and using Morris Water Maze trials. Oral administration of TMP through diet dramatically reduced Aβ accumulation and senile plaques as well as astrogliosis in aged Alzheimer's triple transgenic (3×Tg-AD) mice (See FIGS. 3 and 10). In addition, TMP-induced changes in Aβ pathology were strongly associated with significant improvements in learning and memory (see FIG. 4). Our results are the first to demonstrate the potential benefits of TMP in AD.

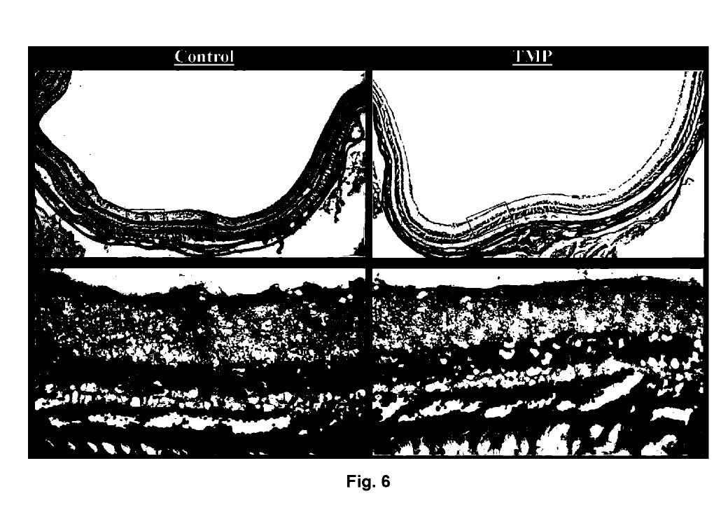

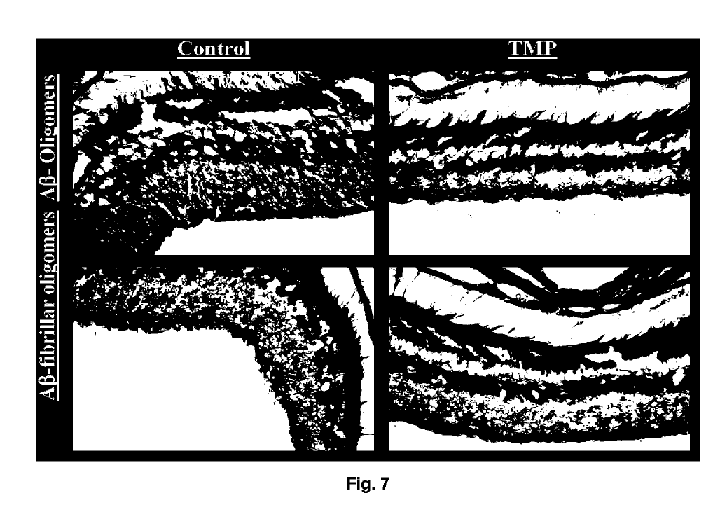

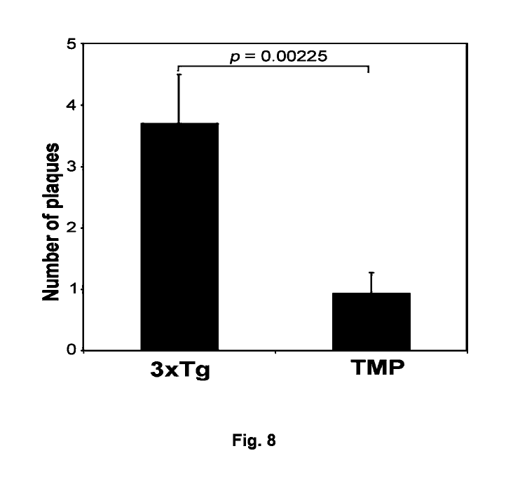

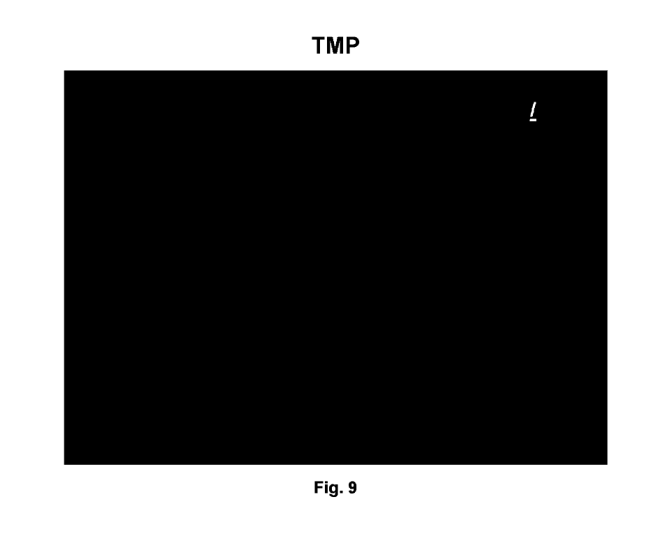

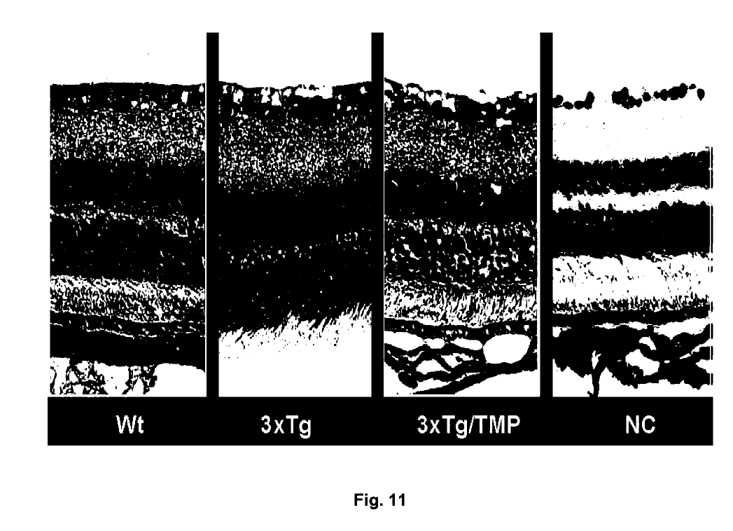

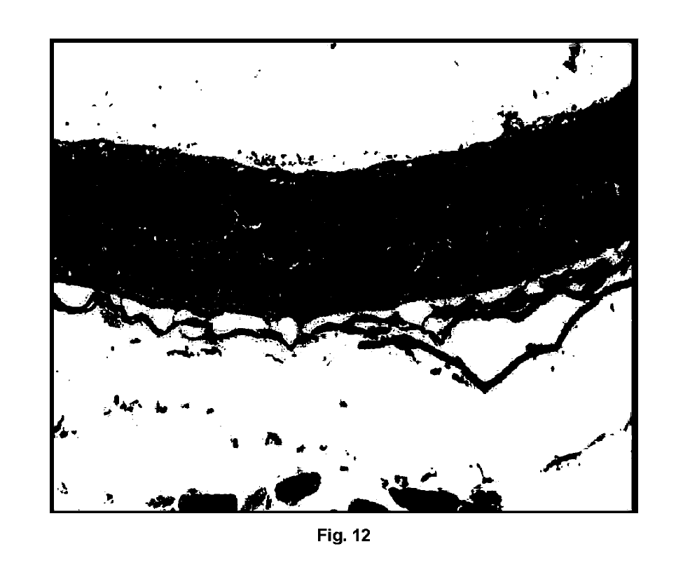

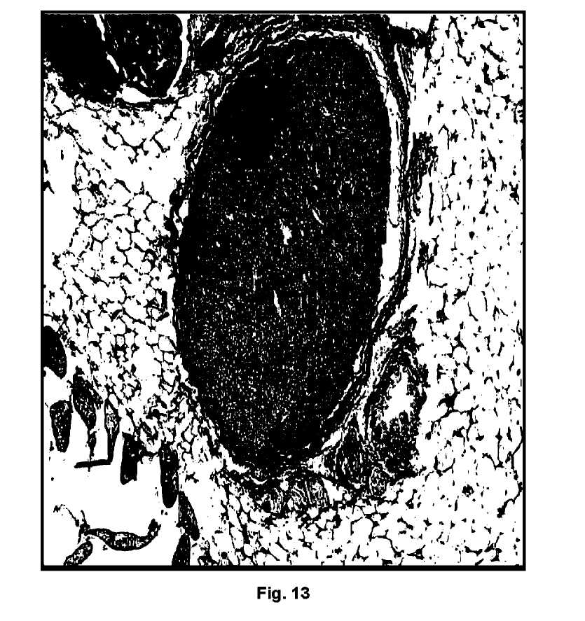

TMP also demonstrated beneficial effects in retinal pathologies associated with disease in 3×Tg-AD mice (see FIGS. 6-9 and 11-13).

### 6.2 Example 2

### Use of Retinal Imaging to Diagnose AD

The following example demonstrates the use of retinal imaging to diagnose AD and to monitor disease progression.

The study used Tg2576 mice that constitutively overexpress APPswe and develop robust Aβ deposits in brain as well as cognitive abnormalities with aging (Hsiao et al., 1996). The pathological changes in the retina of aged mice following different immunization schemes. The Tg2576 mice were immunized with fibrillar Aβ42 and with a prefibrillar oligomer mimetic that gives rise to a prefibrillar oligomer-specific immune response. Both types of immunogens have been shown to be equally effective in reducing plaque deposition and inflammation in Tg2576 mouse brains (Zhou et al., 2005). In this study, another prefibrillar oligomer mimetic antigen was included that uses the islet amyloid polypeptide (IAPP) instead of Aβ, but which gives rise to the same generic prefibrillar oligomer-specific immune response that also recognizes Aβ prefibrillar oligomers (Kayed et al., 2007). While Aβ plaques and microvascular Aβ deposition were observed in the control Tg2576 mouse retinas, mice receiving Aβ and IAPP prefibrillar oligomer vaccinations differentially removed retinal Aβ deposits but exacerbated retinal amyloid angiopathy and inflammation as demonstrated by a significantly enhanced microglial infiltration and astrogliosis.

Materials and Methods

Preparation of Peptides: The Aβ oligomer antigen was prepared from Aβ₁₋₄₀ based on our previously published work (Kayed et al., 2003). Briefly, lyophilized Aβ₁₋₄₀ peptides were resuspended in 50% acetonitrile in water and relyophilized. Soluble prefibrillar oligomers were prepared by dissolving 1.0 mg of peptide in 400 μl of hexafluoroisopropanol for 10˜20 minutes at room temperature. The resultant seedless solution (100 μl) was added to 900 μl of MilliQ H₂O in a siliconized Eppendorf tube. After 10˜20 minutes incubation at room temperature, the samples were centrifuged for 15 minutes at 14,000×g, and the supernatant fraction (pH 2.8˜3.5) was transferred to a new siliconized tube and subjected to a gentle stream of N₂ for 5˜10 minutes to evaporate the hexafluoroisopropanol. The samples were then stirred at 500 rpm using a Teflon-coated micro stir bar for 24-48 hours at 22° C. Oligomers were validated by atomic force microscopy, electron microscopy, and size exclusion chromatography as described previously (Kayed et al., 2003). The Aβ fibril was prepared from Aβ₁₋₄₂, because it is more fibrillogenic as compared with Aβ₁₋₄₀. Aβ₁₋₄₀, predominantly in fibrillary form, was previously studied in a randomized, double-blind, placebo-controlled phase 2a clinical trial, study AN1792(QS-21)-201 by Gilman and colleagues (Gilman et al., 2005). Fibrils were formed by dissolving the lyophilized Aβ₁₋₄₂ in 50% hexafluoroisopropanol and stirred with closed caps for 7 days. Solution is stirred again for 2 days in capped tubes with 20-gauge needle holes on the top to allow the hexafluoroisopropanol to evaporate. This whole preparation was done in a fume food to accelerate evaporation and minimize falling of dust or other foreign material. Fibrils were precipitated by centrifugation for 15 minutes at 14,000 rpm and washed in PBS, and resuspended at 2 mg/ml. For the prefibrillar oligomer antigen, Aβ oligomer (AO) molecular mimic was prepared by conjugating Aβ₁₋₄₀ via a C-terminal thioester to 5-nm colloidal gold as described previously (Kayed and Glabe, 2006). The same procedure was used for prefibrillar IAPP oligomers, and products were stored at 4° C. until used.

Animals and Immunization Schemes: Both Tg2576 mice derived from the Tg(HuAPP695.K670N-M671L)2576 line, a widely used strain created by K. Hsiao (Hsiao et al., 1996) that exhibits robust cerebral extracellular Aβ deposits and cognitive deficits in aged animals, and nontransgenic littermates were used for experiments and controls, respectively. Immunization schemes were conducted in a similar way as described previously (Zhou et al., 2005). Briefly, 4-month-old mice were immunized s.c. with a preparation containing 100 μg of AO, IAPP, or Aβ fibrils (AF) and combined with incomplete Freund's adjuvant (1:1 v/v) once a month for 10 months. Administration of an equivalent volume of PBS with the adjuvant but without any peptide antigen to both wild-type and transgenic mice was used as control. All experimental procedures were performed under protocols approved by the University of California Irvine Institutional Animal Care and Use Committee.

Tissue Preparation: Mice were deeply anesthetized with an overdose of Nembutal (100 mg/kg, i.p.) and perfused transcardially with ice-cold PBS. The eyes were directly collected and fixed overnight with 4% paraformaldehyde in PBS (pH 7.4, 4° C.) following brain removal. Fixed eyes were stored in PBS containing 20% sucrose and 0.05% sodium azide (NaN₃) at 4° C. until use. Following removal of lenses with forceps fixed eyes were cryomolded with OCT. Cross-retinal cryostat sections (12 μm) were thaw-mounted onto “Superfrost plus” glass slides (Fisher, Tustin, Calif.), air-dried for 30 minutes and stored at 4° C. before staining. Brain tissue was fixed in the same way as eyes and was stored in PBS with 0.05% NaN₃. Coronal sections (40 lam) through the dorsal hippocampus were prepared using a Vibratome and were collected in PBS for immunohistochemistry.

Antibodies and Chemicals: The information for all of the antibodies used in this study is summarized in Table 1 (Zhou et al., 2005), (Catania et al., 2009), (Tanzi et al., 1988), (Sisodia and Price, 1995), (Jung et al., 1999), (Parvathy et al., 2001), (Viale et al., 1991), (Sarthy et al., 1991), (Ito et al., 1998), (Goedert et al., 1995), (Uchihara et al., 1997) and (Tan et al., 2002). The avidin-biotin complex kit for diaminobenzidine (DAB) staining and VectaShield fluorescent mounting medium containing 4′,6′-diamidino-2-phenylindole were purchased from Vector Laboratories (Burlingame, Calif.). Unless indicated, all of the chemical reagents used in the experiments were purchased from Sigma-Aldrich (St. Louis, Mo.).

TABLE 1
List of Antibodies Used in the Study

| Antibody and related antigen | Type | Source | Dilution | References |
|---|---|---|---|---|
| OPA1-01132 for human APP | Rabbit | Affinity Bioreagents | 1/100 | Catania et al., 2009 |
| | polyclonal | (Golden, CO) | | |
| BAM01 (6F/3D) for Aβ; cross- | Mouse | Neomarkers | 1/100 | Tanzi et al., 1988; Sisodia |
| reacts with APP | monoclonal | (Fremont, CA) | (retina) | and Price, 1995 |
| | | | 1/500 | |
| | | | (brain) | |
| 6E10 for Aβ; cross-reacts with | Mouse | Covance (Denver, | 1/100 | Zhou et al., 2005; |
| APP | monoclonal | PA) | | Jung et al., 1999 |
| 12F4 for Aβ₄₂ | Mouse | Covance | 1/500 | Parvathy et al., 2001 |
| | monoclonal | | | |
| 5C3 for Aβ₄₀ | Mouse | Assay Designs (Ann | 1/500 | Tanzi et al., 1988; Sisodia |
| | monoclonal | Arbor, MI) | | and Price, 1995 |
| GA5 for GFAP, a marker for | Rabbit | Sigma-Aldrich | 1/500 | Viale et al., 1991; |
| astrocytes or Muller cells | polyclonal | | | Sarthy et al., 1991 |
| CP290A for IBA1, a marker for | Rabbit | Biocare (Concord, | 1/100 | Ito et al., 1998 |
| microglia | polyclonal | CA) | | |
| AT8 for hyperphosphorylated tau | Mouse | Pierce (Rockford, IL) | 1/100 | Goedert et al., 1995 |
| | monoclonal | | | |
| vWF | Rabbit | Sigma-Aldrich | 1/100 | Uchihara et al., 1997 |
| | polyclonal | | | |
| Biotinylated goat anti-mouse IgG | Goat | Vector Laboratories | 1/200 | Tan et al., 2002 |
| | polyclonal | | | |
| Biotinylated goat anti-rabbit IgG | Goat | Vector Laboratories | 1/200 | Tan et al., 2002 |
| | polyclonal | | | |
| Cy3-conjugated goat anti-rabbit | Goat | Sigma-Aldrich | 1/100 | Tan et al., 2002 |
| IgG | polyclonal | | | |
| FITC-conjugated goat anti-rabbit | Goat | Sigma-Aldrich | 1/100 | Tan et al., 2002 |
| IgG | polyclonal | | | |
| Cy3-conjugated donkey anti-mouse | Donkey | Chemicon (Bellerica, | 1/100 | Tan et al., 2002 |
| IgG | polyclonal | MA) | | |

Immunohistochemistry and Quantification: For immunohistochemistry, cross-retinal sections were rehydrated, incubated with 70% formic acid for 5 minutes at room temperature for antigen retrieval, rinsed with PBS, soaked in 3% H₂O₂ for 20 minutes at room temperature to abolish the endogenous peroxidase activity for DAB staining, and/or directly blocked with 5% goat serum in PBS containing 0.1% Triton X-100 and 20 mmol/L L-lysine for 30 minutes at room temperature before incubating with specific primary antibodies overnight at 4° C. The immunoreactivity of Aβ, von Willebrand factor (vWF), which labels vascular endothelial cells, and IBA1 or glial fibrillary acidic protein (GFAP), which label microglia or astrocytes, respectively, was visualized with immunofluorescence microscopy following staining with Cy3-conjugated goat anti-mouse or FITC-conjugated goat anti-rabbit IgG and covered with VectaShield 4′,6′-diamidino-2-phenylindole-containing mounting medium. The immunoreactivity of APP, hyperphosphorylated tau (AT8), and Aβ in selected sections was also detected using the avidin-biotin complex method, followed by staining with DAB and brief counterstaining with hematoxylin. The histological sublayers of retinal cross-sections were identified based on nuclear counterstaining and/or the presence of sclera or pigment epithelium-choroid layer. Brain sections were immunostained free floating following the same procedure as with retinal immunohistochemistry. The results were evaluated by either fluorescence or conventional microscopy with a Leica DM microscope. Images were recorded using a Spot II charge-coupled device camera with equal exposure times for all different groups of tissues. For quantification, the number of Aβ plaques, both Aβ and vWF double-stained capillaries or other vessels, or IBA1-positive cells were counted in each section at a magnification of ×400. Two nonadjacent immunostained sections were examined for each animal. The number of Aβ and vWF double-stained microvessels in a given section was used as a score of amyloid angiopathy.

Congo Red Staining: Congo red staining was performed using a Sigma Congo red kit for amyloid staining according to the manufacturer's protocol with minor modification. Briefly, sections were stained in Mayer's hematoxylin solution for ˜10 seconds, rinsed in tap water, incubated with alkaline sodium chloride for 20 minutes, stained in alkaline Congo red solution for ˜30 minutes, followed by washing in absolute ethanol and mounting. Congo red fluorescence staining was visualized with fluorescence microscopy.

Measurements of the Retinal Thickness: On both sides of each cross-retinal section two images were taken at 200 and 800 μm from the optic nerve at a magnification of ×200. The distance from the surface of the ganglion cell layer to the outside border of the outer nuclear layer was measured using the Adobe Photoshop 7.0 ruler tool. The mean of all four measurements represented the thickness of the retina. Data from three cross-retinal sections were obtained for each animal.

Data Analysis: In all of the graphs that include error bars, the data points represent the means±SEM from all individuals in each group of animals (N=7-9). Where applicable, multiple comparisons were performed by one-way analysis of variance, followed by Student's t-test using Microsoft Excel software. The differences between groups were considered statistically significant when P values were <0.05.

Results

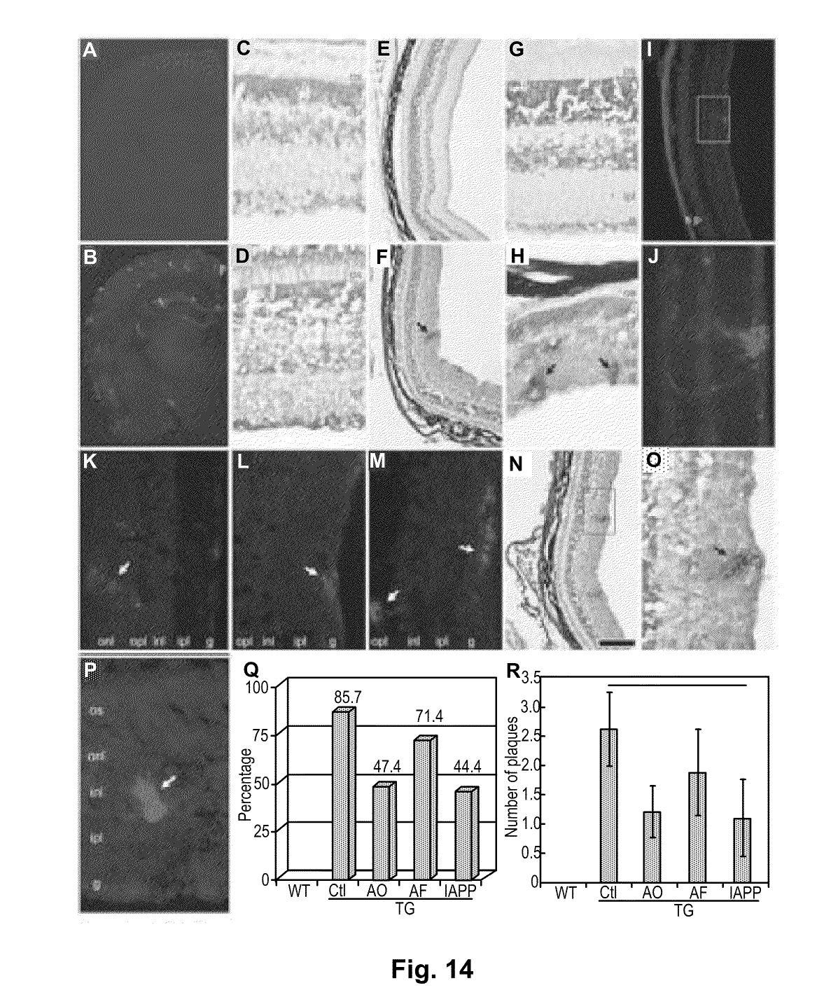

Accumulation of Aβ Deposits and Phosphorylated Tau in the Retinas of Tg2576 Mice: Numerous studies showed abundant expression of APPswe transgene in the nervous system and Aβ plaques in the brain of APPswe transgenic mice older than 12 months (Irizarry et al., 1997). As all of the animals used in this study were 14 months old, immunofluorescence microscopy following 6E10 antibody staining confirmed robust deposition of Aβ plaques in both cortical and hippocampal regions in the Tg2576 mouse brain, whereas an extremely low level of background staining was detected in the wild-type mouse brains (FIG. 14, A and B). To assess expression of the transgene in the retina, the immunoreactivity of human APPswe protein was evaluated following APP-specific antibody staining. Consistent with a recently published observation (Ning et al., 2008), APP immunoreactivity was predominantly detected in the cytoplasm of cells in the ganglion cell layer as well as inner nuclear layer of Tg2576 mice (FIG. 14D). By comparison, a much lower intensity of staining was observed in the corresponding regions of the retina from wild-type mice (FIG. 14C). Interestingly, examination of Aβ immunoreactivity in cross-retinal sections from Tg2576 mice using the human Aβ-specific monoclonal antibody, BAM01 (6F/3D), revealed a remarkable accumulation of Aβ deposits within the retina. Importantly, most senile plaque-like staining patterns were detected from the ganglion cell layer to the outer nuclear layer (FIG. 14, F and H). In rare cases, plaques were found in the photoreceptor outer segment layer and optic nerve as well. To ensure that the plaque staining was not due to nonspecific deposition of DAB, adjacent sections were immunostained with another commonly used monoclonal antibody, 6E10, which demonstrated a staining pattern similar to that obtained with BAM01 (FIG. 14, I and J). Importantly, although both BAM01 and 6E10 antibodies were raised from the N-terminal sequences of Aβ and may cross-react with APP and/or other APP C-terminal fragments on Western blots or enzyme-linked immunosorbent assays, they appear to stain predominantly Aβ in immunohistochemistry (Akiyama et al., 1999) and (Terai et al., 2001) and showed distinct staining patterns from that of APP antibody (FIG. 14D) using the staining protocol as described here. This was further confirmed by a mouse monoclonal antibody that is specific for Aβ₄₀ (FIG. 14K) or Aβ₄₂ (FIG. 14L) in corresponding regions of sections close to those shown in FIG. 14, J and H. Moreover, some of the Aβ deposits were Congo red-positive (FIG. 14, M and P). In contrast, wild-type retinas showed slightly stained background and no plaques (FIG. 14, E and G). Quantification of plaques within the cross-retinal sections indicated about two to three visible plaques detected in the retinas from 85.7% of Tg2576 mice, but no plaques in the retinas of wild-type mice (FIG. 14, Q and R). As previous studies demonstrated hyperphosphorylated tau in the Tg2576 mouse brain (Otth et al., 2002) and (Tomidokoro et al., 2001), whether hyperphosphorylated tau was associated with Aβ deposits in the Tg2576 retina was therefore examined. Adjacent sections were stained with the AT8 antibody, which specifically recognizes hyperphosphorylated tau (Otth et al., 2002) and (Masliah et al., 2001). Remarkably, hyperphosphorylated tau immunoreactivity was detected in regions corresponding to those that were stained by 6E10 for Aβ (FIGS. 14, N and O).

Aβ-Related Vaccinations Reduce Aβ Deposits in the Retinas of Tg2576 Mice: To assess the effect of immunotherapy on retinal deposition of Aβ, immunohistochemistry was performed on retinal sections from Tg2576 mice after a 10-month regime of vaccinations. Two adjacent cross-retinal sections from each animal were stained with either BAM01 or 6E10 and analyzed along with sections from control mice. FIG. 14, P and Q, shows that there was a decrease in plaque-containing retinas and the number of plaques per section that varied with the vaccination immunogen. Particularly, both AO- and IAPP-immunized animals demonstrated a significant reduction in the number of Aβ plaques compared with mice that received PBS adjuvant. The other groups exhibited less pronounced reductions in Aβ deposits that were not statistically significant.

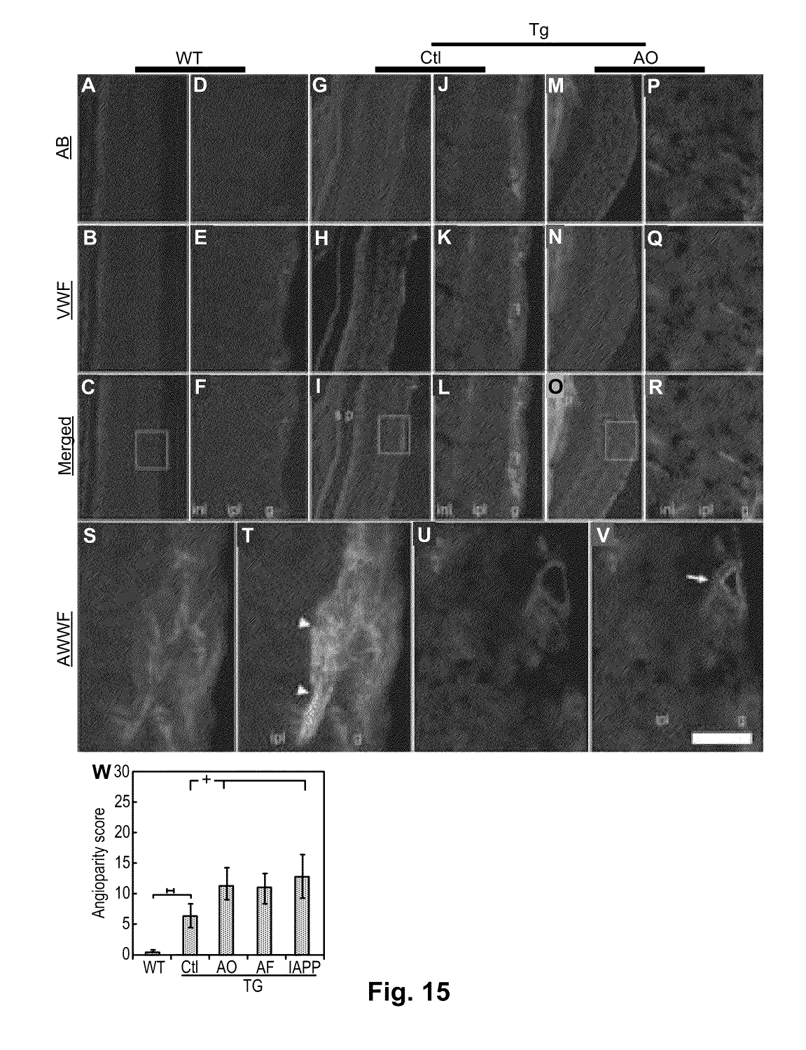

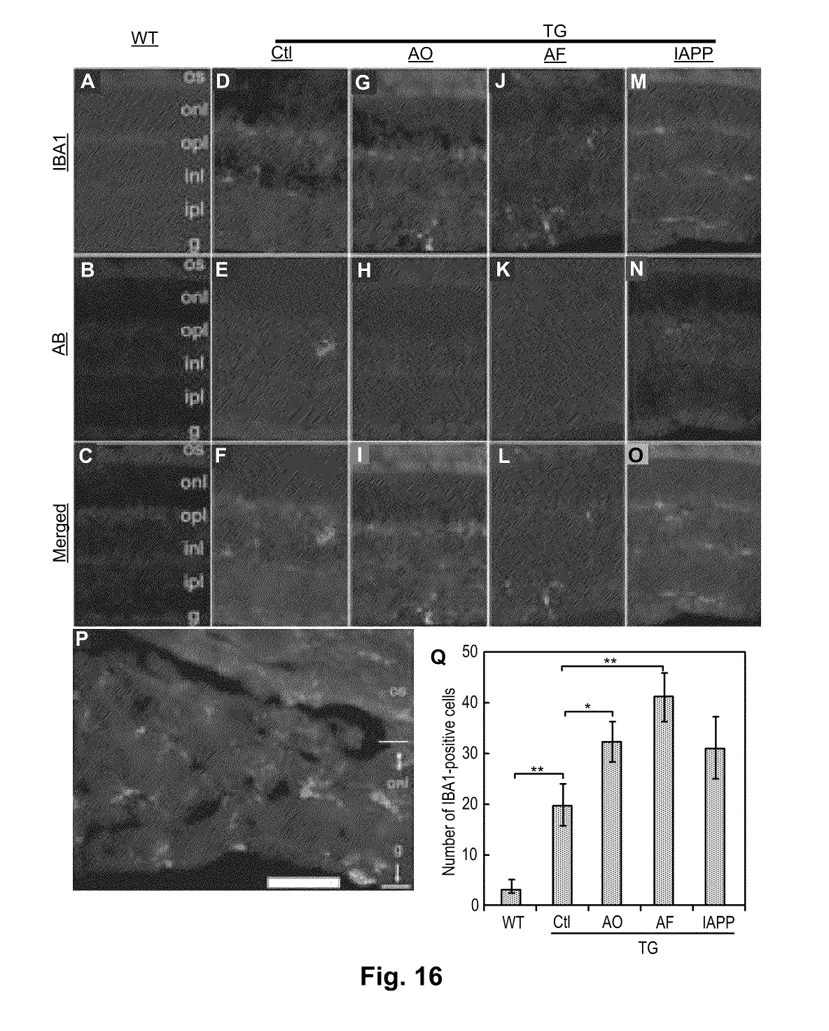

Aβ-Related Vaccinations Exacerbate Retinal Amyloid Angiopathy in Tg2576 Mice: In addition to the accumulation of Aβ plaques in the brain parenchyma, progressive deposition of Aβ peptide in the cerebral microvasculature, that is, cerebral amyloid angiopathy, is a well-known pathological feature of AD (Wisniewski et al., 1997), (Castellani et al., 2004) and (Zhang-Nunes et al., 2006). To examine whether there was any microvascular deposition of Aβ in the retina, that is, retinal amyloid angiopathy, of AD transgenic mice, cross-retinal sections were dual-labeled with both BAM01 and a rabbit polyclonal IgG for vWF, an endothelial cell marker of microvessels. Both Aβ and vWF double-labeled microvessels were quantified and used as retinal amyloid angiopathy scores. FIG. 15 shows representative photomicrographs from wild-type (FIG. 16, A-F) and sham-immunized Tg2576 control mice (FIG. 16, G-L) and mice immunized with AO (FIG. 16, M-R). A similar staining pattern was seen in the mice immunized with either AF or IAPP oligomers as well. Notably, although Aβ immunoreactivity was detected in vWF-labeled microvessels in sections from Tg2576 control mice, even greater Aβ immunoreactivity was present in retinal microvessels from AO-, AF-, or IAPP-immunized mice. Dual-labeled immunoreactivity of Aβ and vWF at a higher magnification demonstrated both intraendothelial (FIG. 15, S and T) and perivascular (FIG. 15, U and V) deposition of Aβ. In contrast, vWF-labeled microvessels in the wild-type mice exhibited no obvious Aβ immunoreactivity (FIG. 15, A-F). Image analysis of sections from groups of animals demonstrated a significant increase in the retinal amyloid angiopathy score of Tg2576 mice compared with wild-type mice (P<0.005), as well as in immunized compared with sham-immunized Tg2576 control mice (FIG. 15W).

Aβ-Related Vaccinations Stimulate Microglial Infiltration and Astrogliosis in the Retinas of Tg2576 Mice: One of the main characteristics accompanying accumulation of Aβ plaques in both human Alzheimer's brain and transgenic mouse models of AD is an enhanced neuroinflammatory response characterized by activation of astrocytes and infiltration of microglia. Therefore, expression of GFAP and IBA1, cell type-specific markers of astrocytes and microglia was examined, respectively, and quantitative image analysis performed to evaluate the degree of microglial infiltration in the immunized animals. FIG. 16 demonstrates representative microphotographs of IBA1- and Aβ-immunoreactivity in the retinas of wild-type, immunized, and sham-immunized mice. Similar to the microvascular deposition of Aβ, there was a dramatic increase in IBA1-immunoreactivity in the retina of Tg2576 mice (FIG. 16, D-O) compared with the wild-type mice (FIG. 16, A-C). Importantly, in some cases, the microglial infiltration was closely associated with disrupted retinal architecture in immunized mice (FIG. 16P), in which all of the different layers from the ganglion cell layer to the outer nuclear layer were damaged and barely distinguished. Quantification of IBA1-positive cells demonstrated a significant increase in microglia in the AD mouse retina (FIG. 16). Importantly, each type of vaccination scheme increased microglial infiltration compared with PBS adjuvant-treated Tg2576 control mice.

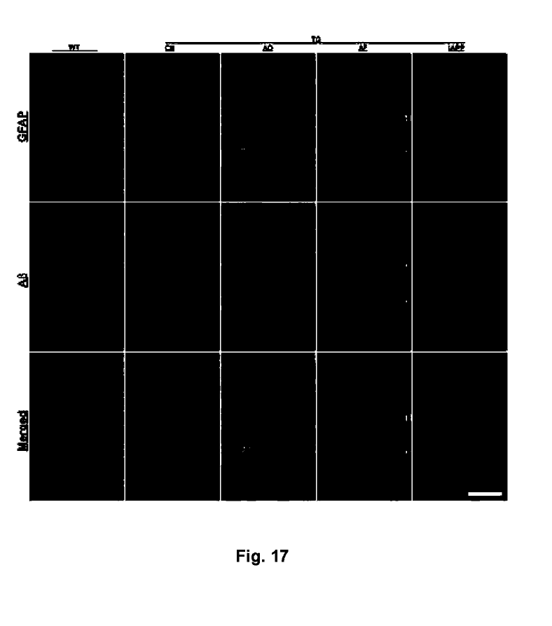

To further examine neuroinflammation in the Alzheimer's retina, astrocytic/Müller cell activation was evaluated based on the immunoreactivity for GFAP. In wild-type mice GFAP immunoreactivity was predominantly limited to the topmost part of the retina or within the ganglion cell layer (FIG. 17). In contrast, the retina of Tg2576 mice exhibited GFAP immunoreactivity not only in the superficial part and the ganglion cell layer as in controls but also in astrocytic processes that penetrated the ganglion cell layer to reach deeper layers. Notably, each of the vaccinations dramatically enhanced this type of staining pattern for GFAP-labeled Müller cell processes (FIG. 17). This observation is consistent with the quantitative changes in microglial infiltration following vaccination and supports the notion that Aβ immunotherapy increases neuroinflammatory responses in the retina.

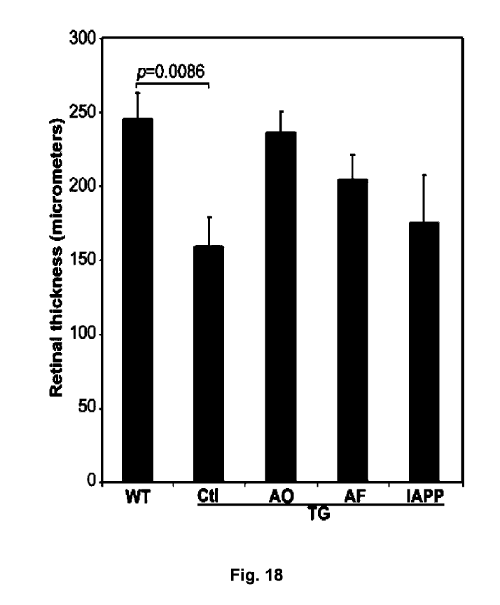

Aβ-Related Vaccinations Differentially Modify Retinal Thickness in Tg2576 Mice: Loss of ganglion cells as well as other retinal neurons has been demonstrated in both human postmortem tissue and mouse models of AD (Blanks et al., 1989), (Ning et al., 2008) and (Miller, 1990). Reduced retinal thickness, a manifestation of cell loss, has been detected in patients with AD (Tetewsky and Duffy, 1999), (Keri et al., 1999) and (Parisi et al., 2001). Because disruption of retinal architecture was evident in Tg2576 mice, the retinal thickness from the surface of the ganglion cell layer to the outside border of the outer nuclear layer was assessed. A significant decrease in the thickness of the retina was found in Tg2576 control mice in comparison with wild-type mice (P=0.0086; FIG. 18). Interestingly, the decrease in retinal thickness was attenuated following a 10-month vaccination protocol, although overall retinal thickness was still reduced compared with wild-type mice.

Discussion

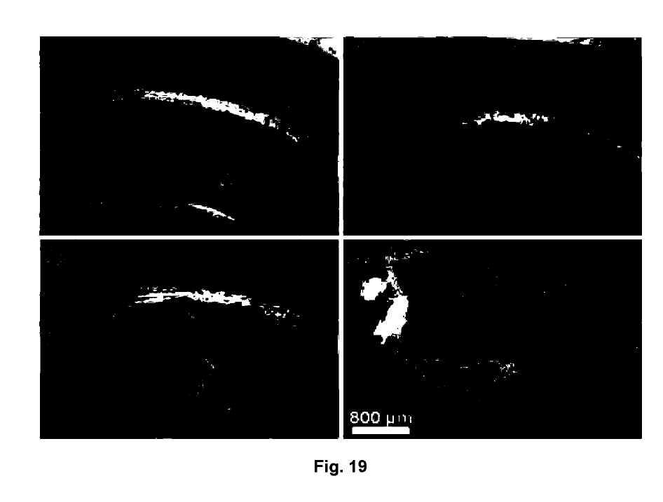

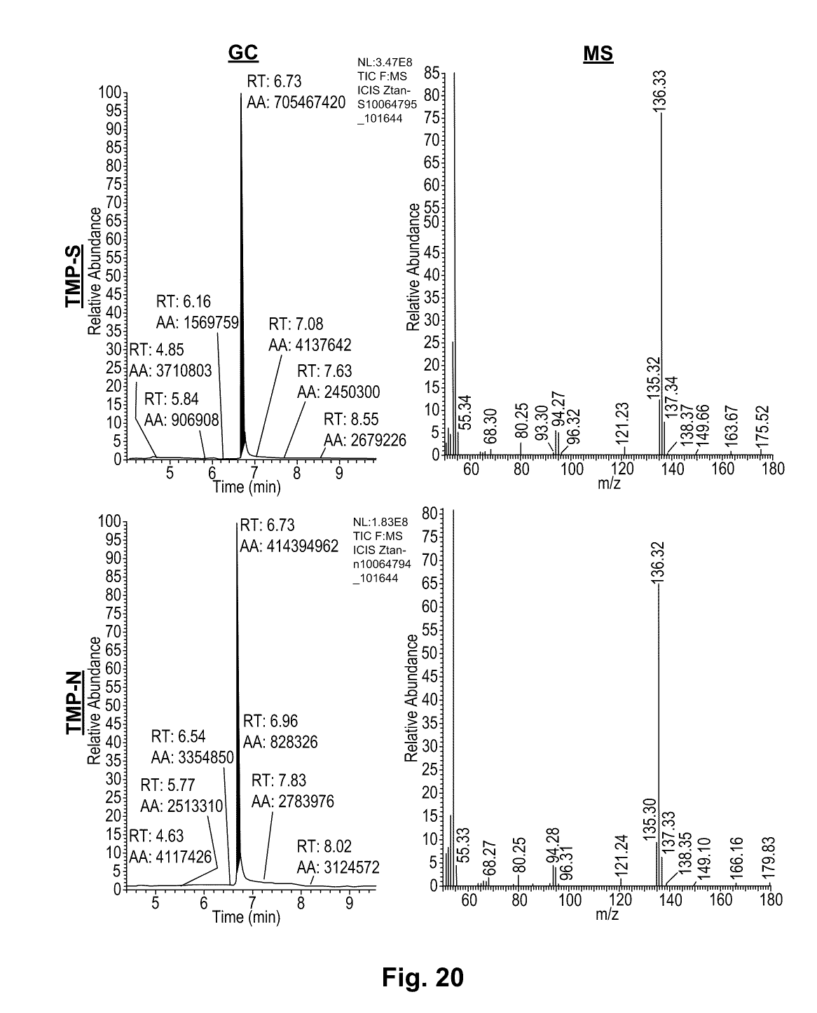

The data presented here are the first demonstration of not only Aβ plaques and amyloid angiopathy in the retina of Tg2576 AD mice but also the effects on retinal pathology of Aβ-related immunotherapy. Detection of the Aβ plaques was confirmed using four different anti-Aβ monoclonal antibodies as well as Congo red fluorescence. Moreover, accumulation of Aβ deposits has been linked with hyperphosphorylation of tau protein. In this study, a hyperphosphorylated form of tau was found to be associated with Aβ plaques within the retina. Vaccination with either a specific prefibrillar oligomeric conformation of Aβ peptide, Aβ fibrils, or human IAPP prefibrillar oligomers differentially reduced retinal Aβ deposits in the Tg2576 mice. The changes mediated by both AOs and IAPP prefibrillar oligomers in the retinas were statistically significant and in agreement with the changes in the hippocampus (FIG. 19) and neocortex. Resembling cerebral amyloid angiopathy in the brain, however, there was robust microvascular Aβ deposition in the retina of AD transgenic mice and a significant increase in the amyloid angiopathy score following each of the immunotherapy regimens. This is consistent with the pathological changes reported in the brain of Alzheimer's transgenic mice following Aβ-based immunotherapy (Petrushina et al., 2008) and (Wilcock et al., 2007). In this regard, the pathological changes in the retina of Alzheimer's mice seem to parallel those in their brain (FIG. 20). These findings of increased microvascular deposition of Aβ support the notion that vaccination-induced antibodies solubilize Aβ plaques and promote Aβ infiltration into the perivascular space (Carare et al., 2008) and (Boche et al., 2008). Notably, Aβ plaques are observed in the retina of other mouse models of AD such as APPswe/PS1ΔE9 (Perez et al., 2009) and 3×Tg-AD mice. Evaluation of retinal amyloid angiopathy may provide an alternative avenue to monitor Aβ clearance mediated by immunotherapy and other therapeutic regimens. Additionally, these results demonstrate increased retinal neuroinflammation in AD transgenic mice as shown by immunoreactivity of both the astrocytic marker, GFAP, and the microglial marker, IBA1.

These results also revealed a significant reduction in retinal thickness in the Tg2576 mice compared with the wild-type mice. This is supported by the observations of retinal degeneration in Tg2576 mice in association with intraneuronal Aβ accumulation (Ning et al., 2008). Therefore, the use of multifaceted behavioral measures may provide a more accurate assessment of cognitive performance in transgenic mouse models of AD. Further studies are warranted to examine correlations between Aβ-associated retinopathy and changes in functional parameters such as the electroretinogram and visual acuity in AD transgenic mice with and without Aβ immunotherapy.

In summary, these results demonstrate Aβ plaques with increased retinal microvascular deposition of Aβ and neuroinflammation in Tg2576 mouse retinas. Aβ and IAPP prefibrillar oligomer vaccinations reduce retinal Aβ deposits but increase retinal microvascular Aβ deposition and exacerbate local neuroinflammation manifested by microglial infiltration and astrogliosis linked with disruption of retinal organization. These results support that a noninvasive imaging modality may be utilized to monitor disease progression and the response to therapeutic interventions.

The present invention is not to be limited in scope by the specific embodiments described herein. Indeed, various modifications of the invention and their equivalents, in addition to those described herein will become apparent to those skilled in the art from the foregoing description and accompanying figures

Various patents, patent applications, and publications are cited herein, the disclosures of which are incorporated by reference in their entirety and for all purposes.

### 6.3 Example 3

### Effects of TMP on Alzheimer's Neuronal Degeneration and Cognitive Impairments

The following example demonstrates that herb-extracted and synthetic forms of TMP exhibit similar neuroprotective effects on neuronal cells against oxidative stress.

### 6.3.1 Purity Analysis of the Two Differently-Sourced TMP Compounds

Two differently-derived TMP were used: (1) an herb-extracted TMP (TMP-N, Advanced Biotech, Paterson, N.J.; Labeled content >99%) and (2) a synthetic TMP (TMP-S, Acros Organics, Morris Plains, N.J.; Labeled content >98%). Gas chromatography-mass spectrometry (GC-MS) analysis revealed that the two differently-sourced TMP compounds were ultra-pure and no evident impurities were detected (FIG. 20).

### 6.3.2 Both Herb-Extracted and Synthetic Forms of TMP Exhibit Similar Neuroprotective Effects on Neuronal Cells Against Oxidative Stress

Treatments of SH-SY5Y human neuroblastoma cells as well as mouse primary neurons with either TMP-N or TMP-S (25-50 μM) significantly increased cell survival following exposure to hydrogen peroxide (10-100 μM). Biochemical analysis confirmed a remarkable reduction of oxidative stress as measured by the production of malondialdehyde (MDA) and hydrogen peroxide in cultured cells. Statistical analysis demonstrated no significant difference for the protective efficacy between two differently-sourced TMP compounds.

### 6.3.3 Both Herb-Extracted and Synthetic Forms of TMP Improve Learning and Memory in AD Mice

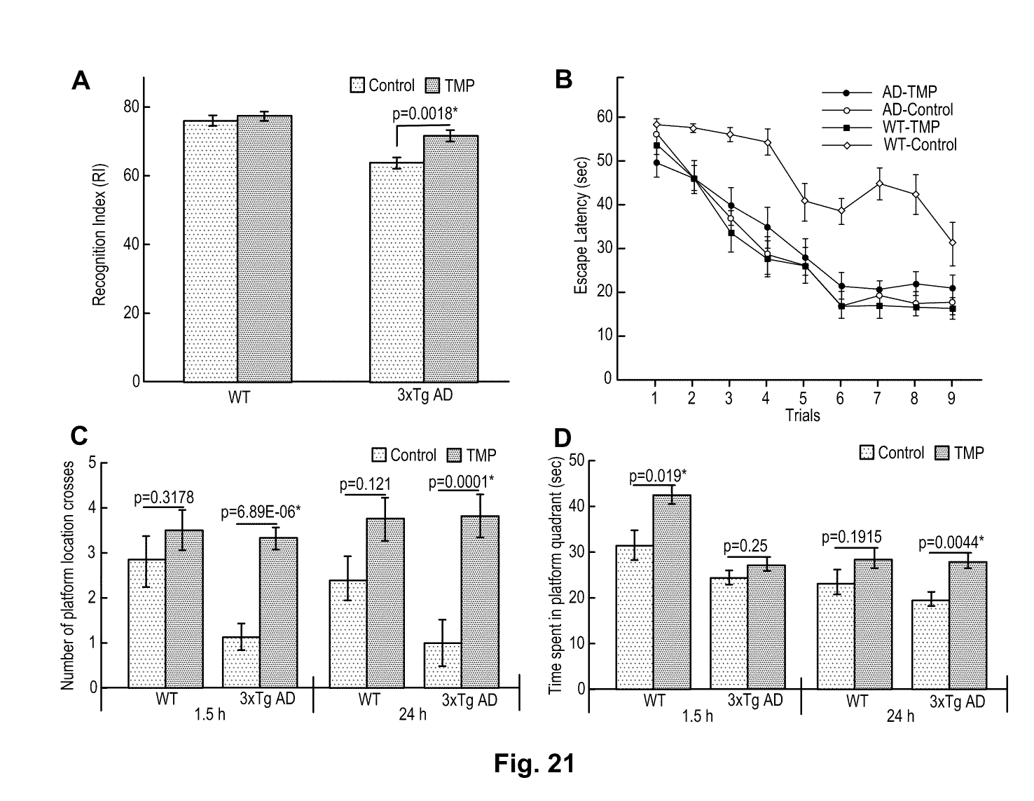

The ability of TMP-N to improve learning and memory in 3×Tg AD mice was assessed using novel object recognition task. The recognition index (RI) score is depicted as: [Time_{N} (in seconds for novel object)/Time_{Total} (in seconds for both old and novel objects)]×100. Starting on day 62, all the mice were trained for 9 days on the Morris water maze (MWM) for the behavior testing. MWM acquisition demonstrated that the treatment significantly improved the performance of 3×Tg-AD mice (FIG. 21).

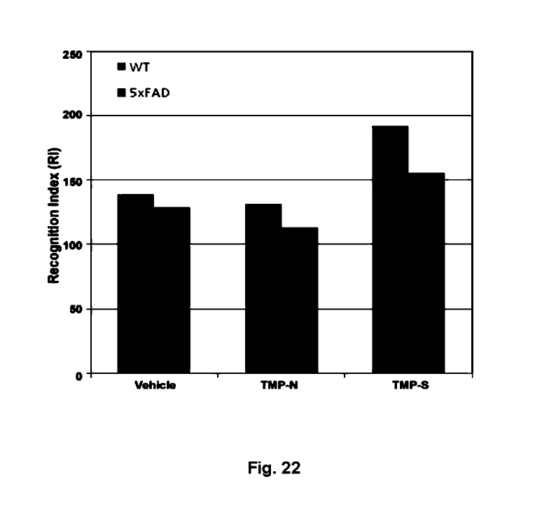

As TMP-N significantly restored learning and memory in aged 3×Tg-AD mice (FIG. 21), the effect of both TMP-N and TMP-S on learning and memory in 5×FAD mice (Oakley et al., J. Neurosci., 26, 10129-40, 2006) was assessed. Wild type littermates were used for comparison. All the mice were fed with normal chow (Vehicle) or containing 300 mg/Kg either TMP-N or TMP-S from postnatal day 61 for exactly 4 months until the day for the completion of behavioral assessments. As demonstrated in FIG. 22, novel object recognition task showed that TMP-S treatment significantly improved memory performance.

### 6.4 Example 4

### Effects of TMP on Alzheimer's Pathology and Retinal Ganglion Cell (RGC) Loss in 5×FAD

The following example demonstrates that TMP reduces beta-Amyloid (Abeta) immunoreactivity in the brains of AD mice and preserves retinal ganglion cells (RGC) in retinas of AD mice.

### 6.4.1 Removal of Beta-Amyloid in 5×FAD Mice by TMP

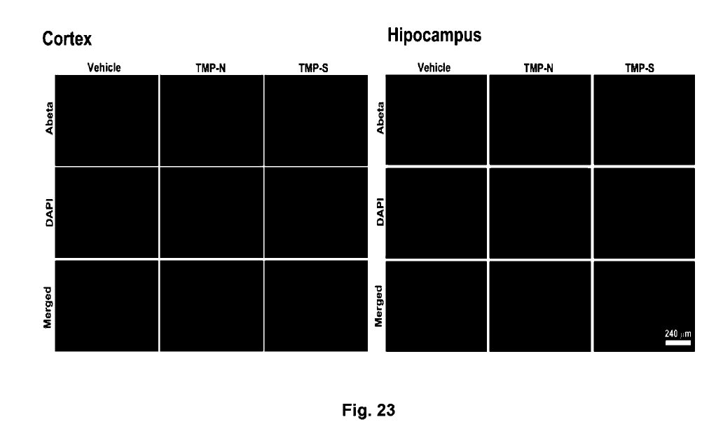

5×FAD mice and wild type littermates (control) were fed with normal chow (Vehicle) or chow containing 300 mg/Kg of either TMP-N or TMP-S (see Section 6.3.1) from postnatal day 61 for exactly 4 months until the day of analysis. Animal tissues were perfused with 1×PBS followed by overnight fixation in 4% paraformaldehyde in cold room. Vibrotome sections (˜50 μm thick) were immunostained with 6E10 antibody, which recognizes all forms of Abeta, and visualized by fluorescence microscopy. FIG. 23 demonstrates reduction Abeta immunoreactivity in the brain of AD mice following TMP treatment. Although behavioral tests showed some negative effects of TMP-N treatment on memory performance, Abeta deposits were decreased in both cortical and hippocampal regions following treatment with TMP-N.

### 6.4.2 TMP Treatment Prevents Retinal Ganglion Cell (RGC) Loss in 5×FAD Mice

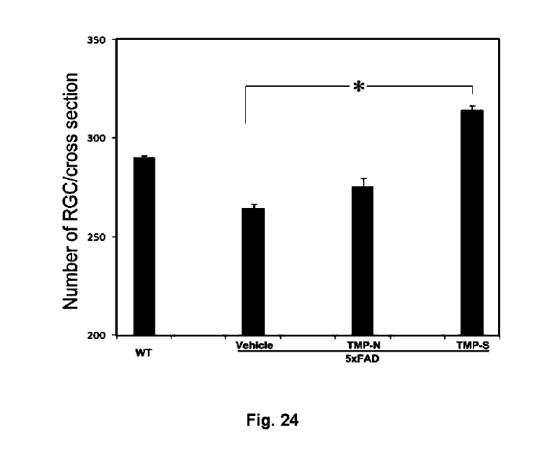

5×FAD mice and wild type littermates (control) were fed with normal chow (Vehicle) or chow containing 300 mg/Kg of either TMP-N or TMP-S (see Section 6.3.1) from postnatal day 61 for exactly 4 months until the day of analysis. RGC cells were quantified in cross retinal sections as described in Liu et al., American Journal of Pathology, 2009. As shown in FIG. 24, TMP-S (see Section 6.3.1) treatment significantly preserved RGC in 5×FAD mouse retinas, while TMP-N treatment slightly preserved RGC in 5×FAD mouse retinas.

Abeta accumulation is believed to play an important role in the retinal degeneration in glaucoma. Thus, the neuroprotective efficacy of TMP-S on RGC suggests that TMP should be used for the treatment of glaucoma.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### REFERENCES

Adams T B, Doull J, Feron V J, Goodman J I, Marnett L J, Munro I C, et al. The FEMA GRAS assessment of pyrazine derivatives used as flavor ingredients. Flavor and Extract Manufacturers Association. Food Chem. Toxicol. 2002; 40:429-451.
Akiyama H, Mori H, Saido T, Kondo H, Ikeda K, McGeer P L: Occurrence of the diffuse amyloid β-protein (Aβ) deposits with numerous Aβ-containing glial cells in the cerebral cortex of patients with Alzheimer's disease. Glia 1999, 25:324-331.
Almaas R, Saugstad O D, Pleasure D, Rootwelt T. Neuronal formation of free radicals plays a minor role in hypoxic cell death in human NT2-N neurons. Pediatr Res. 2002; 51:136-143.
Amaratunga A, Fine R E. Generation of amyloidogenic C-terminal fragments during rapid axonal transport in vivo of beta-amyloid precursor protein in the optic nerve. J Biol. Chem. 1995; 270:17268-17272.
Areias F M, Rego A C, Oliveira C R, Seabra R M. Antioxidant effect of flavonoids after ascorbate/Fe(2+)-induced oxidative stress in cultured retinal cells. Biochem Pharmacol. 2001; 62:111-118.
Ates O, Cayli S R, Yucel N, Altinoz E, Kocak A, Durak M A, et al. Central nervous system protection by resveratrol in streptozotocin-induced diabetic rats. J Clin Neurosci. 2007; 14:256-260.
Baluchnejadmojarad T, Roghani M. Effect of naringenin on intracerebroventricular streptozotocin-induced cognitive deficits in rat: a behavioral analysis. Pharmacology. 2006; 78:193-197.
Bard F, Cannon C, Barbour R, Burke R L, Games D, Grajeda H, Guido T, Hu K, Huang J, Johnson-Wood K, Khan K, Kholodenko D, Lee M, Lieberburg I, Motter R, Nguyen M, Soriano F, Vasquez N, Weiss K, Welch B, Seubert P, Schenk D, Yednock T: Peripherally administered antibodies against amyloid β-peptide enter the central nervous system and reduce pathology in a mouse model of Alzheimer disease. Nat Med 2000, 6:916-919.
Berisha F, Feke G T, Trempe C L, McMeel J W, Schepens CL: Retinal abnormalities in early Alzheimer's disease. Invest Ophthalmol V is Sci 2007, 48:2285-2289.
Blanks J C, Hinton D R, Sadun A A, Miller C A: Retinal ganglion cell degeneration in Alzheimer's disease. Brain Res 1989, 501:364-372.
Blanks J C, Schmidt S Y, Torigoe Y, Porrello K V, Hinton D R, Blanks R H: Retinal pathology in Alzheimer's disease. II. Regional neuron loss and glial changes in GCL. Neurobiol Aging 1996, 17:385-395.
Boche D, Zotova E, Weller R O, Love S, Neal J W, Pickering R M, Wilkinson D, Holmes C, Nicoll JA: Consequence of Aβ immunization on the vasculature of human Alzheimer's disease brain. Brain 2008, 131:3299-3310.
Bouayed J, Rammal H, Dicko A, Younos C, Soulimani R. Chlorogenic acid, a polyphenol from Prunus domestica (Mirabelle), with coupled anxiolytic and antioxidant effects. J Neurol Sci. 2007; 262:77-84.
Bredesen D E, Rao R V, Mehlen P. Cell death in the nervous system. Nature. 2006; 443:796-802.
Brown R E, Wong A A: The influence of visual ability on learning and memory performance in 13 strains of mice. Learn Mem 2007, 14:134-144.
Bureau G, Longpre F, Martinoli M G. Resveratrol and quercetin, two natural polyphenols, reduce apoptotic neuronal cell death induced by neuroinflammation. J Neurosci Res. 2008; 86:403-410.
Butter C M, Trobe J D, Foster N L, Berent S: Visual-spatial deficits expalin visual symptoms in Alzheimer's disease. Am J Ophthalmol 1996, 122:97-105.
Carare R O, Bernardes-Silva M, Newman T A, Page A M, Nicoll J A, Perry V H, Weller R O: Solutes, but not cells, drain from the brain parenchyma along basement membranes of capillaries and arteries: significance for cerebral amyloid angiopathy and neuroimmunology. Neuropathol Appl Neurobiol 2008, 34:131-144.
Casson R J. Possible role of excitotoxicity in the pathogenesis of glaucoma. Clin Experiment Ophthalmol. 2006; 34:54-63.
Castellani R J, Smith M A, Perry G, Friedland R P: Cerebral amyloid angiopathy: major contributor or decorative response to Alzheimer's disease pathogenesis. Neurobiol Aging 2004, 25:599-602; discussion 603-594.
Catania C, Sotiropoulos I, Silva R, Onofri C, Breen K C, Sousa N, Almeida O F: The amyloidogenic potential and behavioral correlates of stress. Mol Psychiatry 2009, 14:95-105.
Chan D, Crutch S J, Warrington E K: A disorder of colour perception associated with abnormal colour after-images: a defect of the primary visual cortex. J Neurol Neurosurg Psychiatry 2001, 71:515-517.
Chang Y, Hsiao G, Chen S H, Chen Y C, Lin J H, Lin K H, et al. Tetramethylpyrazine suppresses HIF-1alpha, TNF-alpha, and activated caspase-3 expression in middle cerebral artery occlusion-induced brain ischemia in rats. Acta Pharmacologica Sinica. 2007; 28:327-333.
Chao H M, Lin D E, Chang Y, Hsu W M, Lee S M, Lee F L, et al. Ferulic acid, but not tetramethylpyrazine, significantly attenuates retinal ischemia/reperfusion-induced alterations by acting as a hydroxyl radical scavenger. J Ocul Pharmacol Ther. 2008; 24:461-472.
Chen K J, Chen K. Ischemic stroke treated with Ligusticum chuanxiong. Chin Med J (Engl) 1992; 105:870-873.
Chiou G C, Xu X R. Effects of some natural flavonoids on retinal function recovery after ischemic insult in the rat. J Ocul Pharmacol Ther. 2004; 20:107-113.
Chiou G C, Yan H Y, Lei X L, Li B H, Shen Z F. Ocular and cardiovascular pharmacology of tetramethylpyrazine isolated from Ligusticum wallichii Franch. Zhongguo Yao Li Xue Bao. 1991; 12:99-104.
Cho J, Lee H K. Wogonin inhibits excitotoxic and oxidative neuronal damage in primary cultured rat cortical cells. Eur J Pharmacol. 2004; 485:105-110.
Cho J, Lee H K. Wogonin inhibits ischemic brain injury in a rat model of permanent middle cerebral artery occlusion. Biol Pharm Bull. 2004; 27:1561-1564.
Cho J Y, Kim I S, Jang Y H, Kim A R, Lee S R. Protective effect of quercetin, a natural flavonoid against neuronal damage after transient global cerebral ischemia. Neurosci Lett. 2006; 404:330-335.
Dajas F, Rivera F, Blasina F, Arredondo F, Echeverry C, Lafon L, et al. Cell culture protection and in vivo neuroprotective capacity of flavonoids. Neurotox Res. 2003; 5:425-432.
Danesh-Meyer H V, Birch H, Ku J Y, Carroll S, Gamble G: Reduction of optic nerve fibers in patients with Alzheimer disease identified by laser imaging. Neurology 2006, 67:1852-1854.
Dehmelt L, Halpain S. Actin and microtubules in neurite initiation: are MAPs the missing link? J Neurobiol. 2004; 58:18-33.
Dong L P, Wang T Y. Effects of puerarin against glutamate excitotoxicity on cultured mouse cerebral cortical neurons. Zhongguo Yao Li Xue Bao. 1998; 19:339-342.
Fan et al., 2006 L. H. Fan, K. Z. Wang, B. Cheng, C. S. Wang and X. Q. Dang, Anti-apoptotic and neuroprotective effects of tetramethylpyrazine following spinal cord ischemia in rabbits, BMC Neurosci. 7 (2006), p. 48.
Fan L H, Wang K Z, Cheng B, Wang C S, Dang X Q. Anti-apoptotic and neuroprotective effects of Tetramethylpyrazine following spinal cord ischemia in rabbits. BMC Neurosci. 2006; 7:48.
Forette F, Orgogozo J M: Clinical effects of Aβ immunization (AN1792) in patients with AD in an interrupted trial. Neurology 2005, 64:1553-1562.
Frederikse P H, Ren X O: Lens defects and age-related fiber cell degeneration in a mouse model of increased AβPP gene dosage in Down syndrome. Am J Pathol 2002, 161:1985-1990.
Gao B T. The effects of ligustrazine, aspirin and beta-histine on platelet aggregation in patients with acute ischemic stroke. Zhonghua Shen Jing Jing Shen Ke Za Zhi. 1989; 22:148-151.
Garcia-Alloza M, Ferrara B J, Dodwell S A, Hickey G A, Hyman B T, Bacskai B J: A limited role for microglia in antibody mediated plaque clearance in APP mice. Neurobiol Dis 2007, 28:286-292.
Gilman S, Koller M, Black R S, Jenkins L, Griffith S G, Fox N C, Eisner L, Kirby L, Rovira M B, Goedert M, Jakes R, Vanmechelen E: Monoclonal antibody AT8 recognises tau protein phosphorylated at both serine 202 and threonine 205. Neurosci Lett 1995, 189:167-169.
Goldstein L E, Muffat J A, Chemy R A, Moir R D, Ericsson M H, Huang X, Mavros C, Coccia J A, Faget K Y, Fitch K A, Masters C L, Tanzi R E, Chylack L T, Jr, Bush A I: Cytosolic β-amyloid deposition and supranuclear cataracts in lenses from people with Alzheimer's disease. Lancet 2003, 361:1258-1265.
Gottlieb M, Leal-Campanario R, Campos-Esparza M R, Sanchez-Gomez M V, Alberdi E, Arranz A, et al. Neuroprotection by two polyphenols following excitotoxicity and experimental ischemia. Neurobiol Dis. 2006; 23:374-386.
Greenberg S M: Cerebral amyloid angiopathy: prospects for clinical diagnosis and treatment. Neurology 1998, 51:690-694.
Gu J W, Hasuo H, Takeya M, Akasu T. Effects of emodin on synaptic transmission in rat hippocampal CA1 pyramidal neurons in vitro. Neuropharmacology. 2005; 49:103-111.
Hale G, Good M: Impaired visuospatial recognition memory but normal object novelty detection and relative familiarity judgments in adult mice expressing the APPswe Alzheimer's disease mutation. Behav Neurosci 2005, 119:884-891.
Hanneken A, Lin F F, Johnson J, Maher P. Flavonoids protect human retinal pigment epithelial cells from oxidative-stress-induced death. Invest Ophthalmol V is Sci. 2006; 47:3164-3177.
Hardy J, Selkoe D J: The amyloid hypothesis of Alzheimer's disease: progress and problems on the road to therapeutics. Science 2002, 297:353-356.
Hashimoto et al., 2001 Y. Hashimoto, T. Niikura, H. Tajima, T. Yasukawa, H. Sudo, Y. Ito, Y. Kita, M. Kawasumi, K. Kouyama, M. Doyu, G. Sobue, T. Koide, S. Tsuji, J. Lang, K. Kurokawa and I. Nishimoto, A rescue factor abolishing neuronal cell death by a wide spectrum of familial Alzheimer's disease genes and Abeta, Proc. Natl. Acad. Sci. U.S.A. 98 (2001), pp. 6336-6341.
Head E, Lott I T: Down syndrome and β-amyloid deposition. Curr Opin Neurol 2004, 17:95-100.
Heneka M T, O'Banion M K: Inflammatory processes in Alzheimer's disease. J Neuroimmunol 2007, 184:69-91.
Hinton D R, Sadun A A, Blanks J C, Miller C A: Optic-nerve degeneration in Alzheimer's disease. N Engl J Med 1986, 315:485-487.
Ho J W, Jie M. Pharmacological activity of cardiovascular agents from herbal medicine. Cardiovasc Hematol Agents Med. Chem. 2007; 5:273-277.
Hsiao K, Chapman P, Nilsen S, Eckman C, Harigaya Y, Younkin S, Yang F, Cole G: Correlative memory deficits, Aβ elevation, and amyloid plaques in transgenic mice. Science 1996, 274:99-102.
Huang K J, Williams W M. The pharmacology of Chinese herbs. Boca Raton: CRC; 1993.
Huang X, Ren P, Wen A D, Wang L L, Zhang L, Gao F. Pharmacokinetics of traditional Chinese syndrome and recipe: a hypothesis and its verification (I) World J Gastroenterol. 2000; 6:384-391.
Ibarretxe G, Sanchez-Gomez M V, Campos-Esparza M R, Alberdi E, Matute C. Differential oxidative stress in oligodendrocytes and neurons after excitotoxic insults and protection by natural polyphenols. Glia. 2006; 53:201-211.
Irizarry M C, McNamara M, Fedorchak K, Hsiao K, Hyman B T: APPSw transgenic mice develop age-related Aβ deposits and neuropil abnormalities, but no neuronal loss in CA1. J Neuropathol Exp Neurol 1997, 56:965-973.
Iseri P K, Altinas O, Tokay T, Yuksel N: Relationship between cognitive impairment and retinal morphological and visual functional abnormalities in Alzheimer disease. J Neuroophthalmol 2006, 26:18-24.
Ishii Y, Kwong J M, Caprioli J. Retinal ganglion cell protection with geranylgeranylacetone, a heat shock protein inducer, in a rat glaucoma model. Invest Ophthalmol V is Sci. 2003; 44:1982-1992.
Ito D, Imai Y, Ohsawa K, Nakajima K, Fukuuchi Y, Kohsaka S: Microglia-specific localisation of a novel calcium binding protein, Ibal. Brain Res Mol Brain Res 1998, 57:1-9.
Jagatha B, Mythri R B, Vali S, Bharath M M. Curcumin treatment alleviates the effects of glutathione depletion in vitro and in vivo: therapeutic implications for Parkinson's disease explained via in silico studies. Free Radic Biol Med. 2008; 44:907-917.
Jung S S, Gauthier S, Cashman N R: β-Amyloid precursor protein is detectable on monocytes and is increased in Alzheimer's disease. Neurobiol Aging 1999, 20:249-257.
Kao et al., 2006 T. K. Kao, Y. C. Ou, J. S. Kuo, W. Y. Chen, S. L. Liao, C. W. Wu, C. J. Chen, N. N. Ling, Y. H. Zhang and W. H. Peng, Neuroprotection by tetramethylpyrazine against ischemic brain injury in rats, Neurochem. Int. 48 (2006), pp. 166-176.
Katz B, Rimmer S: Ophthalmologic manifestations of Alzheimer's disease. Surv Ophthalmol 1989, 34:31-43.
Kayed R, Glabe C G: Conformation-dependent anti-amyloid oligomer antibodies. Methods Enzymol 2006, 413:326-344.
Kayed R, Head E, Sarsoza F, Saing T, Cotman C W, Necula M, Margol L, Wu J, Breydo L, Thompson J L, Rasool S, Gurlo T, Butler P, Glabe C G: Fibril specific, conformation dependent antibodies recognize a generic epitope common to amyloid fibrils and fibrillar oligomers that is absent in prefibrillar oligomers. Mol Neurodegener 2007, 2:18.
Kayed R, Head E, Thompson J L, McIntire T M, Milton S C, Cotman C W, Glabe C G: Common structure of soluble amyloid oligomers implies common mechanism of pathogenesis. Science 2003, 300:486-489.
Kazantsev A G. Cellular pathways leading to neuronal dysfunction and degeneration. Drug News Perspect. 2007; 20:501-509.
Keri S, Antal A, Kalman J, Janka Z, Benedek G: Early visual impairment is independent of the visuocognitive and memory disturbances in Alzheimer's disease. Vision Res 1999, 39:2261-2265
Kobayashi M S, Han D, Packer L. Antioxidants and herbal extracts protect HT-4 neuronal cells against glutamate-induced cytotoxicity. Free Radic Res. 2000; 32:115-124.
Kook D, Wolf A H, Yu A L, Neubauer A S, Priglinger S G, Kampik A, et al. The protective effect of quercetin against oxidative stress in the human RPE in vitro. Invest Ophthalmol Vis Sci. 2008; 49:1712-1720.
Kramerov A A, Saghizadeh M, Pan H, Kabosova A, Montenarh M, Ahmed K, et al. Expression of protein kinase CK2 in astroglial cells of normal and neovascularized retina. Am J Pathol. 2006; 168:1722-1736.
Laabich A, Manmoto C C, Kuksa V, Leung D W, Vissvesvaran G P, Karliga I, et al. Protective effects of myricetin and related flavonols against A2E and light mediated-cell death in bovine retinal primary cell culture. Exp Eye Res. 2007; 85:154-165.
Lakshminarayanan V, Lagrave J, Kean M L, Dick M, Shankle R: Vision in dementia: contrast effects. Neurol Res 1996, 18:9-15.
Le Cudennec C, Faure A, Ly M, Delatour B: One-year longitudinal evaluation of sensorimotor functions in APP751SL transgenic mice. Genes Brain Behav 2008, 7(Suppl 1):83-91.
Lee H J, Noh Y H, Lee D Y, Kim Y S, Kim K Y, Chung Y H, et al. Baicalein attenuates 6-hydroxydopamine-induced neurotoxicity in SH-SY5Y cells. Eur J Cell Biol. 2005; 84:897-905.
Li J, Xiong X, Liu Y. Protective effect of paeoniflorin against optic nerve crush. Journal of Huazhong University of Science and Technology. 2007; 27:650-652.
Liao M H, Wu C C, Yen M H. Beneficial effects of tetramethylpyrazine, an active constituent of Chinese herbs, on rats with endotoxemia. Proc Natl Sci Counc Repub China B. 1998; 22:46-54.
Liao S L, Kao T K, Chen W Y, Lin Y S, Chen S Y, Raung S L, et al. Tetramethylpyrazine reduces ischemic brain injury in rats. Neurosci Lett. 2004; 372:40-45.
Lin F, Xin Y, Wang J, Ma L, Liu J, Liu C, et al. Puerarin facilitates Ca(2+)-induced Ca(2+) release triggered by KCl-depolarization in primary cultured rat hippocampal neurons. Eur J Pharmacol. 2007; 570:43-49.
Lin H J, Lai C C, Lee Chao P D, Fan S S, Tsai Y, Huang S Y, et al. Aloe-emodin metabolites protected N-methyl-d-aspartate-treated retinal ganglion cells by Cu—Zn superoxide dismutase. J Ocul Pharmacol Ther. 2007; 23:152-171.
Liu H Q, Zhang W Y, Luo X T, Ye Y, Zhu X Z. Paeoniflorin attenuates neuroinflammation and dopaminergic neurodegeneration in the MPTP model of Parkinson's disease by activation of adenosine A1 receptor. Br J Pharmacol. 2006; 148:314-325.
Liu J, Jin D Z, Xiao L, Zhu X Z. Paeoniflorin attenuates chronic cerebral hypoperfusion-induced learning dysfunction and brain damage in rats. Brain Res. 2006; 1089:162-170.
Liu S F, Cai Y N, Evans T W, McCormack D G, Barer G R, Barnes P J. Ligustrazine is a vasodilator of human pulmonary and bronchial arteries. Eur J Pharmacol. 1990; 191:345-350.
Liu Y H, Liu Y F, Guo X X. Current studies on anti-endotoxic chemical components of traditional Chinese medicine in China. Acta Pharmacologica Sinica. 2001; 22:1071-1077.
Lopez-Sanchez C, Martin-Romero F J, Sun F, Luis L, Samhan-Arias A K, Garcia-Martinez V, et al. Blood micromolar concentrations of kaempferol afford protection against ischemia/reperfusion-induced damage in rat brain. Brain Res. 2007; 1182:123-137.
Lou Y Q, Zhang H, Cao X, Chen M L. The pharmacokinetics and disposition of tetramethylpyrazine phosphate in dogs and rats. Yao Xue Xue Bao. 1986; 21:481-487.
Mandal M N, Patlolla J M, Zheng L, Agbaga M P, Tran J T, Wicker L, et al. Curcumin protects retinal cells from light- and oxidant stress-induced cell death. Free Radic Biol Med. 2009; 46:672-679.
Marambaud P, Zhao H, Davies P. Resveratrol promotes clearance of Alzheimer's disease amyloid-beta peptides. J Biol. Chem. 2005; 280:37377-37382.
Masliah E, Sisk A, Mallory M, Games D: Neurofibrillary pathology in transgenic mice overexpressing V717F β-amyloid precursor protein. J Neuropathol Exp Neurol 2001, 60:357-368.
Matteucci A, Frank C, Domenici M R, Balduzzi M, Paradisi S, Carnovale-Scalzo G, et al. Curcumin treatment protects rat retinal neurons against excitotoxicity: effect on N-methyl-d-aspartate-induced intracellular Ca(2+) increase. Exp Brain Res. 2005; 167:641-648.
Mattson M P: Pathways towards and away from Alzheimer's disease. Nature 2004, 430:631-639.
Mattson M P. Neuronal life-and-death signaling, apoptosis, and neurodegenerative disorders. Antioxid Redox Signal. 2006; 8:1997-2006.
McKinnon S J, Lehman D M, Kerrigan-Baumrind L A, Merges C A, Pease M E, Kerrigan D F, Ransom N L, Tahzib N G, Reitsamer H A, Levkovitch-Verbin H, Quigley H A, Zack D J: Caspase activation and amyloid precursor protein cleavage in rat ocular hypertension. Invest Ophthalmol Vis Sci 2002, 43:1077-1087.
Mendola J D, Cronin-Golomb A, Corkin S, Growdon J H: Prevalence of visual deficits in Alzheimer's disease. Optom V is Sci 1995, 72:155-167.
Miller N R: Alzheimer's disease, optic neuropathy, and selective ganglion cell damage. Ophthalmology 1990, 97:7-8.
Monsonego A, Weiner H L: Immunotherapeutic approaches to Alzheimer's disease. Science 2003, 302:834-838.
Mozaffarieh M, Grieshaber M C, Orgul S, Flammer J. The potential value of natural antioxidative treatment in glaucoma. Surv Ophthalmol. 2008; 53:479-505.
Nagaki Y, Hayasaka S, Kadoi C, Nakamura N, Hayasaka Y. Effects of scutellariae radix extract and its components (baicalein, baicalin, and wogonin) on the experimental elevation of aqueous flare in pigmented rabbits. Jpn J Ophthalmol. 2001; 45:216-220.
Nakajima Y, Shimazawa M, Mishima S, Hara H. Water extract of propolis and its main constituents, caffeoylquinic acid derivatives, exert neuroprotective effects via antioxidant actions. Life Sci. 2007; 80:370-377.
Nakamura N, Hayasaka S, Zhang X Y, Nagaki Y, Matsumoto M, Hayasaka Y, et al. Effects of baicalin, baicalein, and wogonin on interleukin-6 and interleukin-8 expression, and nuclear factor-kappab binding activities induced by interleukin-1beta in human retinal pigment epithelial cell line. Exp Eye Res. 2003; 77:195-202.
Nelson G A, Edward D P, Wilensky J T: Ocular amyloidosis and secondary glaucoma. Ophthalmology 1999, 106:1363-1366.
Ni J W, Matsumoto K, Watanabe H. Tetramethylpyrazine improves spatial cognitive impairment induced by permanent occlusion of bilateral common carotid arteries or scopolamine in rats. Jpn J Pharmacol. 1995; 67:137-141.
Ning A, Cui J, To E, Ashe K H, Matsubara J: Amyloid-beta deposits lead to retinal degeneration in a mouse model of Alzheimer disease. Invest Ophthalmol V is Sci 2008, 49:5136-5143.
Occelli E, Mariani L, Fontanella L, Corsico N. Substances with central nervous system activity. Derivatives of octahydro-1,4-dihydroxypyrrolo[1,2-a]pyrazine-6-carboxylic acids. Farmaco [Sci] 1984; 39:718-738.
Ohnishi R, Ito H, Iguchi A, Shinomiya K, Kamei C, Hatano T, et al. Effects of chlorogenic acid and its metabolites on spontaneous locomotor activity in mice. Biosci Biotechnol Biochem. 2006; 70:2560-2563.
Ono K, Yoshiike Y, Takashima A, Hasegawa K, Naiki H, Yamada M. Potent anti-amyloidogenic and fibril-destabilizing effects of polyphenols in vitro: implications for the prevention and therapeutics of Alzheimer's disease. J Neurochem. 2003; 87:172-181.
Otth C, Concha I I, Arendt T, Stieler J, Schliebs R, Gonzalez-Billault C, Maccioni R B: AβPP induces cdk5-dependent tau hyperphosphorylation in transgenic mice Tg2576. J Alzheimers Dis 2002, 4:417-430.
Oyama Y, Fuchs P A, Katayama N, Noda K. Myricetin and quercetin, the flavonoid constituents of Ginkgo biloba extract, greatly reduce oxidative metabolism in both resting and Ca(2+)-loaded brain neurons. Brain Res. 1994; 635:125-129.
Ozaki Y. Antiinflammatory effect of tetramethylpyrazine and ferulic acid. Chem Pharm Bull (Tokyo) 1992; 40:954-956.
Pache M, Smeets C H, Gasio P F, Savaskan E, Flammer J, Wirz-Justice A, Kaiser H J: Colour vision deficiencies in Alzheimer's disease. Age Ageing 2003, 32:422-426.
Pang P K, Shan J J, Chiu K W. Tetramethylpyrazine, a calcium antagonist. Planta Med. 1996; 62:431-435.
Paquet C, Boissonnot M, Roger F, Dighiero P, Gil R, Hugon J: Abnormal retinal thickness in patients with mild cognitive impairment and Alzheimer's disease. Neurosci Lett 2007, 420:97-99.
Parisi V, Restuccia R, Fattapposta F, Mina C, Bucci M G, Pierelli F: Morphological and functional retinal impairment in Alzheimer's disease patients. Clin Neurophysiol 2001, 112:1860-1867.
Park Y H, Xu X R, Chiou G C. Structural requirements of flavonoids for increment of ocular blood flow in the rabbit and retinal function recovery in rat eyes. J Ocul Pharmacol Ther. 2004; 20:189-200.
Parker J A, Arango M, Abderrahmane S, Lambert E, Tourette C, Catoire H, et al. Resveratrol rescues mutant polyglutamine cytotoxicity in nematode and mammalian neurons. Nat. Genet. 2005; 37:349-350.
Parvathy S, Davies P, Haroutunian V, Purohit D P, Davis K L, Mohs R C, Park H, Moran T M, Chan J Y, Buxbaum J D: Correlation between Aβx-40-. Aβx-42-, and Aβx-43-containing amyloid plaques and cognitive decline. Arch Neurol 2001, 58:2025-2032.
Peng W, Duan S F. Effects of ligustrazine controlled release capsule in chronic pulmonary heart disease. J Tongji Med. Univ. 1991; 11:101-105.
Perez S E, Lumayag S, Kovacs B, Mufson E J, Xu S: β-Amyloid deposition and functional impairment in the retina of the APPswe/PS1ΔE9 transgenic mouse model of Alzheimer's disease. Invest Ophthalmol Vis Sci 2009, 50:793-800.
Petrushina I, Ghochikyan A, Mkrtichyan M, Mamikonyan G, Movsesyan N, Ajdari R, Vasilevko V, Karapetyan A, Lees A, Agadjanyan M G, Cribbs D H: Mannan-Aβ28 conjugate prevents Aβ-plaque deposition, but increases microhemorrhages in the brains of vaccinated Tg2576 (APPsw) mice. J Neuroinflammation 2008, 5:42.
Pu F, Mishima K, Irie K, Motohashi K, Tanaka Y, Orito K, et al. Neuroprotective effects of quercetin and rutin on spatial memory impairment in an 8-arm radial maze task and neuronal death induced by repeated cerebral ischemia in rats. J Pharmacol Sci. 2007; 104:329-334.
Qi X, Ackermann C, Sun D, Liu R, Sheng M, Hou H. The prediction of plasma and brain levels of 2, 3, 5, 6-tetramethylpyrazine following transdermal application. AAPS PharmSci. 2002; 4:E46.
Qi X, Ackermann C, Sun D, Sheng M, Hou H. Physicochemical characterization and percutaneous delivery of 2, 3, 5, 6-tetramethylpyrazine. Int J Pharm. 2003; 253:177-183.
Quach N, Chan T, Lu T A, Schreiber S S, Tan Z. Induction of DNA repair proteins, Ref-1 and XRCC1, in adult rat brain following kainic acid-induced seizures. Brain Res. 2005; 1042:236-240.
Rathore P, Dohare P, Varma S, Ray A, Sharma U, Jaganathanan N R, et al. Curcuma oil: reduces early accumulation of oxidative product and is anti-apoptogenic in transient focal ischemia in rat brain. Neurochem Res. 2008; 33:1672-1682.
Ren P, Huang X, Jiang Y P. Effect of sijunzi decoction on motilin and pharmacokinetic characteristics of tetramethylpyrazine in rat model of spleen deficiency syndrome. Zhongguo Zhong Xi Yi Jie He Za Zhi. 1997; 17:45-47.
Sadun A A, Borchert M, DeVita E, Hinton D R, Bassi C J: Assessment of visual impairment in patients with Alzheimer's disease. Am J Ophthalmol 1987, 104:113-120.
Salinska E, Danysz W, Lazarewicz J W. The role of excitotoxicity in neurodegeneration. Folia Neuropathol. 2005; 43:322-339.
Samhan-Arias A K, Martin-Romero F J, Gutierrez-Merino C. Kaempferol blocks oxidative stress in cerebellar granule cells and reveals a key role for reactive oxygen species production at the plasma membrane in the commitment to apoptosis. Free Radic Biol Med. 2004; 37:48-61.
Sarthy P V, Fu M, Huang J: Developmental expression of the glial fibrillary acidic protein (GFAP) gene in the mouse retina. Cell Mol Neurobiol 1991, 11:623-637.
Schonlau F, Rohdewald P. Pycnogenol for diabetic retinopathy. A review. Int Ophthalmol. 2001; 24:161-171.
Sexton A, McDonald M, Cayla C, Thiemermann C, Ahluwalia A. 12-Lipoxygenase-derived eicosanoids protect against myocardial ischemia/reperfusion injury via activation of neuronal TRPV1. FASEB J. 2007; 21:2695-2703.
Shieh D E, Liu L T, Lin C C. Antioxidant and free radical scavenging effects of baicalein, baicalin and wogonin. Anticancer Res. 2000; 20:2861-2865.
Shih et al., 2002 Y. H. Shih, S. L. Wu, W. F. Chiou, H. H. Ku, T. L. Ko and Y. S. Fu, Protective effects of tetramethylpyrazine on kainate-induced excitotoxicity in hippocampal culture, Neuroreport 13 (2002), pp. 515-519.
Shih Y H, Wu S L, Chiou W F, Ku H H, Ko T L, Fu Y S. Protective effects of tetramethylpyrazine on kainate-induced excitotoxicity in hippocampal culture. NeuroReport. 2002; 13:515-519.
Shukla P K, Khanna V K, Ali M M, Khan M Y, Srimal R C. Anti-ischemic effect of curcumin in rat brain. Neurochem Res. 2008; 33:1036-1043.
Silva B A, Dias A C, Ferreres F, Malva J O, Oliveira C R. Neuroprotective effect of H. perforatum extracts on beta-amyloid-induced neurotoxicity. Neurotox Res. 2004; 6:119-130.
Sisodia S S, Price D L: Role of the β-amyloid protein in Alzheimer's disease. FASEB J 1995, 9:366-370.
Soliman K F, Mazzio E A. In vitro attenuation of nitric oxide production in C6 astrocyte cell culture by various dietary compounds. Proc Soc Exp Biol Med. 1998; 218:390-397.
Spadea L, Balestrazzi E. Treatment of vascular retinopathies with Pycnogenol. Phytother Res. 2001; 15:219-223.
Sperk G. Kainic acid seizures in the rat. Prog Neurobiol. 1994; 42:1-32.
Strenn K, Dal-Bianco P, Weghaupt H, Koch G, Vass C, Gottlob I: Pattern electroretinogram and luminance electroretinogram in Alzheimer's disease. J Neural Transm Suppl 1991, 33:73-80.
Tan Z, Levid J, Schreiber S S. Increased expression of Fas (CD95/APO-1) in adult rat brain after kainate-induced seizures. NeuroReport. 2001; 12:1979-1982.
Tan Z, Sankar R, Shin D, Sun N, Liu H, Wasterlain C G, et al. Differential induction of p53 in immature and adult rat brain following lithium-pilocarpine status epilepticus. Brain Res. 2002; 928:187-193.
Tan Z, Sankar R, Tu W, Shin D, Liu H, Wasterlain C G, Schreiber S S: Immunohistochemical study of p53-associated proteins in rat brain following lithium-pilocarpine status epilepticus. Brain Res 2002, 929:129-138.
Tan Z, Tu W, Schreiber S S. Downregulation of free ubiquitin: a novel mechanism of p53 stabilization and neuronal cell death. Brain Res Mol Brain Res. 2001; 91:179-188.
Tang X. Effect of ligustrazine on proliferative glomerulonephritis. Zhong Yao Cai. 2003; 26:611-612.
Tanzi R E, McClatchey A I, Lamperti E D, VIIIa-Komaroff L, Gusella J F, Neve R L: Protease inhibitor domain encoded by an amyloid protein precursor mRNA associated with Alzheimer's disease. Nature 1988, 331:528-530.
Teng Y, Cui H, Yang M, Song H, Zhang Q, Su Y, et al. Protective effect of puerarin on diabetic retinopathy in rats. Mol Biol Rep. 2008; 36:1129-1133.
Terai K, Iwai A, Kawabata S, Tasaki Y, Watanabe T, Miyata K, Yamaguchi T: β-Amyloid deposits in transgenic mice expressing human β-amyloid precursor protein have the same characteristics as those in Alzheimer's disease. Neuroscience 2001, 104:299-310.
Tetewsky S J, Duffy C J: Visual loss and getting lost in Alzheimer's disease. Neurology 1999, 52:958-965.
Tomidokoro Y, Harigaya Y, Matsubara E, Ikeda M, Kawarabayashi T, Shirao T, Ishiguro K, Okamoto K, Younkin S G, Shoji M: Brain Aβ amyloidosis in APPsw mice induces accumulation of presenilin-1 and tau. J Pathol 2001, 194:500-506.
Trushina E, McMurray C T. Oxidative stress and mitochondrial dysfunction in neurodegenerative diseases. Neuroscience. 2007; 145:1233-1248.
Tsai C S, Ritch R, Schwartz B, Lee S S, Miller N R, Chi T, Hsieh F Y: Optic nerve head and nerve fiber layer in Alzheimer's disease. Arch Ophthalmol 1991, 109:199-204.
Tsai T H, Liang C. Pharmacokinetics of tetramethylpyrazine in rat blood and brain using microdialysis. Int J Pharm. 2001; 216:61-66.
Tsai T Y, Wu S N, Liu Y C, Wu A Z, Tsai Y C Inhibitory action of L-type Ca2+ current by paeoniflorin, a major constituent of peony root, in NG108-15 neuronal cells. Eur J Pharmacol. 2005; 523:16-24.
Tsuda T, Sugaya A, Ohguchi H, Kishida N, Sugaya E. Protective effects of peony root extract and its components on neuron damage in the hippocampus induced by the cobalt focus epilepsy model. Exp Neurol. 1997; 146:518-525.
Veurink G, Liu D, Taddei K, Perry G, Smith M A, Robertson T A, et al. Reduction of inclusion body pathology in ApoE-deficient mice fed a combination of antioxidants. Free Radic Biol Med. 2003; 34:1070-1077.
Viale G, Gambacorta M, Coggi G, Dell'Orto P, Milani M, Doglioni C: Glial fibrillary acidic protein immunoreactivity in normal and diseased human breast. Virchows Arch A Pathol Anat Histopathol 1991, 418:339-348.
Wang C, Zhang D, Ma H, Liu J. Neuroprotective effects of emodin-8-O-beta-d-glucoside in vivo and in vitro. Eur J Pharmacol. 2007; 577:58-63.
Wang C N, Chi C W, Lin Y L, Chen C F, Shiao Y J. The neuroprotective effects of phytoestrogens on amyloid beta protein-induced toxicity are mediated by abrogating the activation of caspase cascade in rat cortical neurons. J Biol. Chem. 2001; 276:5287-5295.
Wang G J. The change in the nailfold microcirculation in patients with acute cerebral thrombosis treated with ligustrazine. Zhonghua Shen Jing Jing Shen Ke Za Zhi. 1984; 17:121-124.
Wang S Y, Wang H H, Chi C W, Chen C F, Liao J F. Effects of baicalein on beta-amyloid peptide-(25-35)-induced amnesia in mice. Eur J Pharmacol. 2004; 506:55-61.
Wang X F, Zhao M Q. Ligustrazine and Salvia miltiorrhiza injection solution in complementary therapy of pregnancy-induced hypertension: clinical analysis of 60 cases. Di Yi Jun Yi Da Xue Xue Bao. 2003; 23:969-971.
Whittaker K W, Burdon M A, Shah P: Visual field loss and Alzheimer's disease. Eye 2002, 16:206-208.
Wilcock D M, Jantzen P T, Li Q, Morgan D, Gordon M N: Amyloid-β vaccination, but not nitro-nonsteroidal anti-inflammatory drug treatment, increases vascular amyloid and microhemorrhage while both reduce parenchymal amyloid. Neuroscience 2007, 144:950-960.
Wisniewski H M, Vorbrodt A W, Wegiel J: Amyloid angiopathy and blood-brain barrier changes in Alzheimer's disease. Ann NY Acad Sci 1997, 826:161-172.
Wu P H, Shen Y C, Wang Y H, Chi C W, Yen J C. Baicalein attenuates methamphetamine-induced loss of dopamine transporter in mouse striatum. Toxicology. 2006; 226:238-245.
Xu H, Shi D Z, Guan C Y. Clinical application and pharmacological actions of ligustrazine. Zhongguo Zhong Xi Yi Jie He Za Zhi. 2003; 23:376-379.
Xu X, Zhang S, Zhang L, Yan W, Zheng X. The Neuroprotection of puerarin against cerebral ischemia is associated with the prevention of apoptosis in rats. Planta Med. 2005; 71:585-591.
Xu X H, Zhao T Q. Effects of puerarin on d-galactose-induced memory deficits in mice. Acta Pharmacologica Sinica. 2002; 23:587-590.
Xuan B, Zhou Y H, Yang R L, Li N, Min Z D, Chiou G C. Improvement of ocular blood flow and retinal functions with puerarin analogs. J Ocul Pharmacol Ther. 1999; 15:207-216.
Yan F, Luo R. Effects of ligustrazine on blood vessels and blood components. Zhong Yao Cai. 2002; 25:143-145.
Yang J N, Chen J M, Luo L, Lin S C, Li D, Hu S X. Tetramethylpyrazine protected photoreceptor cells of rats by modulating nuclear translocation of NF-kappaB. Acta Pharmacologica Sinica. 2005; 26:887-892.
Yang L, Sun H L, Wu L M, Guo X J, Dou H, Tso M O, et al. Baicalein reduces inflammatory process in a rodent model of diabetic retinopathy. Invest Ophthalmol V is Sci. 2009; 50:2319-2327.
Yang Z, Zhang Q, Ge J, Tan Z. Protective effects of tetramethylpyrazine on rat retinal cell cultures. Neurochem Int. 2008; 52:1176-1187.
Yankner B A, Lu T, Loerch P: The aging brain. Annu Rev Pathol 2008, 3:41-66
Yankner B A, Lu T: Amyloid β-protein toxicity and the pathogenesis of Alzheimer's disease. J Biol Chem 2009, 284:4755-4759.
Zbarsky V, Datla K P, Parkar S, Rai D K, Aruoma O I, Dexter D T. Neuroprotective properties of the natural phenolic antioxidants curcumin and naringenin but not quercetin and fisetin in a 6-OHDA model of Parkinson's disease. Free Radic Res. 2005; 39:1119-1125.
Zhang C, Wang S Z, Zuo P P, Cui X, Cai J. Protective effect of tetramethylpyrazine on learning and memory function in d-galactose-lesioned mice. Chin Med Sci J. 2004; 19:180-184.
Zhang Z, Wei T, Hou J, Li G, Yu S, Xin W. Iron-induced oxidative damage and apoptosis in cerebellar granule cells: attenuation by tetramethylpyrazine and ferulic acid. Eur J Pharmacol. 2003; 467:41-47.
Zhang Z, Wei T, Hou J, Li G, Yu S, Xin W. Tetramethylpyrazine scavenges superoxide anion and decreases nitric oxide production in human polymorphonuclear leukocytes. Life Sci. 2003; 72:2465-2472.
Zhang Z H, Yu S Z, Wang Z T, Zhao B L, Hou J W, Yang F J, et al. Scavenging effects of tetramethylpyrazine on active oxygen free radicals. Zhongguo Yao Li Xue Bao. 1994; 15:229-231.
Zhang-Nunes S X, Maat-Schieman M L, van Duinen S G, Roos R A, Frosch M P, Greenberg S M: The cerebral β-amyloid angiopathies: hereditary and sporadic. Brain Pathol 2006, 16:30-39.
Zheng L T, Ock J, Kwon B M, Suk K. Suppressive effects of flavonoid fisetin on lipopolysaccharide-induced microglial activation and neurotoxicity. Int Immunopharmacol. 2008; 8:484-494.
Zhou J, Fonseca M I, Kayed R, Hernandez I, Webster S D, Yazan O, Cribbs D H, Glabe C G, Tenner A J: Novel Aβ peptide immunogens modulate plaque pathology and inflammation in a murine model of Alzheimer's disease. J Neuroinflammation 2005, 2:28.

1. A method for treating a degenerative disorder of the central nervous system (CNS) in a human patient comprising administering an effective amount of tetramethylpyrazine (TMP) to a human patient diagnosed with a degenerative disorder of the CNS.

2. A method for treating a human patient at risk for developing a degenerative disorder of the CNS comprising administering an effective amount of tetramethylpyrazine (TMP) to a human patient diagnosed has being at risk for developing a degenerative disorder of the CNS.

3. The method of claim 1, wherein the CNS disorder is Alzheimer's Disease (AD).

4. The method of claim 1, wherein the CNS disorder is Parkinson's Disease (PD).

5. The method of claim 1, wherein the CNS disorder is Huntington's Disease (HD).

6. The method of claim 1, wherein the CNS disorder is mild cognitive impairment (MCI).

7. The method of claim 1, wherein the CNS disorder is traumatic brain injury.

8. The method of claim 1, wherein TMP is administered at a dose from about 0.01 mg/kg to about 300 mg/kg.

9. The method of claim 8, wherein TMP is administered at a dose of about 50.0 mg/kg.

10. The method of claim 8, wherein TMP is administered at a dose of about 300 mg/kg.

11. The method of claim 1, wherein TMP is administered as part of a treatment regimen that includes one or more additional therapies for the treatment of the CNS disorder.

12. The method of claim 11, wherein the additional therapy is an agent selected from the group consisting of Aricept, Razadyne, Namenda, Exelon and Cognex.

13. The method of claim 1, wherein TMP is administered orally, intraocularly, or subcutaneously.

14. The method of claim 1, wherein the administration of TMP results in improved retinal pathologies in the human patient.

15. A method for diagnosing a human patient as having a degenerative CNS disorder comprising performing retinal imaging on the patient, wherein the presence of one or more pathological changes, relative to an earlier retinal image of the patient or to a normal retina, in the human patient indicates that the patient has a degenerative CNS disorder.

16. A method for determining the efficacy of a therapeutic regimen for the treatment of a degenerative CNS disorder comprising performing retinal imaging on a patient prior to the onset of the therapeutic regimen and during or at the completion of the therapeutic regimen, wherein an improvement in retinal pathology indicates that the therapy is effective.

17. The method of claim 15, wherein the CNS disorder is Alzheimer's Disease (AD).

18. The method of claim 15, wherein the CNS disorder is Parkinson's Disease (PD).

19. The method of claim 15, wherein the CNS disorder is Huntington's Disease (HD).

20. The method of claim 15, wherein the CNS disorder is mild cognitive impairment (MCI).

21. The method of claim 15, wherein the CNS disorder is traumatic brain injury.

22. The method of claim 15, wherein the CNS disorder is glaucoma.

23. The method of claim 15, wherein the pathology measured is presence of Aβ plaques, amyloid angiopathy or retinal thickness.

24. The method of claim 17, wherein the pathology measured is the presence of Aβ plaques, amyloid angiopathy or retinal thickness.

25. The method of claim 16 wherein the therapeutic regimen comprises the administration of TMP.

26. The method of claim 17 wherein the therapeutic regimen comprises the administration of TMP.

27. A method for treating glaucoma in a human patient comprising administering an effective amount of tetramethylpyrazine (TMP) to a human patient diagnosed with glaucoma.

28. A method for treating a human patient at risk for developing glaucoma comprising administering an effective amount of tetramethylpyrazine (TMP) to a human patient diagnosed has being at risk for developing glaucoma.

29. The method of claim 1, wherein the TMP compound is administered in a formulation wherein the TMP compound is the sole active agent.
